(19) United States
(12) Patent Application Publication
(10) Pub. No.: US 2023/0050694 A1
(43) Pub. Date: Feb. 16, 2023

(54) SYSTEMS AND METHODS FOR DIGITAL IMAGING AND COMMUNICATIONS IN MEDICINE FILE PROCESSING

(71) Applicant: WUHAN UNITED IMAGING HEALTHCARE CO., LTD., Wuhan (CN)

(72) Inventors: Yonghui WU, Wuhan (CN); Hao LUO, Wuhan (CN)

(73) Assignee: WUHAN UNITED IMAGING HEALTHCARE CO., LTD., Wuhan (CN)

(21) Appl. No.: 17/819,609

(22) Filed: Aug. 12, 2022

(30) Foreign Application Priority Data
Aug. 12, 2021 (CN) 202110923288.X
Dec. 28, 2021 (CN) 202111631678.6

(51) Int. Cl. G16H 30/20; G16H 30/40

(52) U.S. Cl. CPC G16H 30/20; G16H 30/40

(57) ABSTRACT

The present disclosure provides systems and methods for digital imaging and communications in medicine (DICOM) file processing. The methods may include receiving a request for processing a DICOM file. The DICOM file may include data of metadata and pixel data. The methods may also include parsing at least part of the metadata of the DICOM file. The methods may further include writing the data of the DICOM file to one or more data streams based on the parsed metadata.

### CROSS-REFERENCE TO RELATED APPLICATIONS

This application claims priority of Chinese Application No. 202110923288.X, filed on Aug. 12, 2021, and Chinese Application No. 202111631678.6, filed on Dec. 28, 2021, the contents of which are hereby incorporated by reference.

### TECHNICAL FIELD

The present disclosure generally relates to the field of medical file processing, and more particularly, relates to systems and methods for digital imaging and communications in medicine (DICOM) file processing.

### BACKGROUND

Medical information systems (e.g., picture archiving and communication systems (PACS)) usually support the DICOM protocol. The DICOM protocol may be implemented by C++, C#, or java, and cannot fully support the reception and/or transmission of a large file. Generally, when receiving or sending a medical image file that follows the DICOM protocol, the PACS will save the full DICOM file into its memory or generate a local cache version of the DICOM file. DICOM files generated by different medical imaging devices are different. Many of the DICOM files are larger than 500 M (sometimes reaching several Gigabytes) which makes them difficult to be handled by traditional DICOM systems.

Sometimes, if several DICOM files need to be fully read through the memory, with the increase of the amount of the DICOM files and the frequencies of processing the DICOM files, the memory consumption would surge, leading to a slow response of the medical information system. If the DICOM files are read through a local cache instead of the memory, the consumption of input/output (I/O) read and write resources would increase, resulting in poor performance of the whole system. To improve the response time and performance, the system requires powerful hardware which would bring higher costs.

Transmission of large DICOM files is further prone to suffer from network transmission timeout and low network performance, resulting in file transmission failure. Therefore, it is desirable to provide systems and methods for DICOM file processing with improved performance for transmission of large DICOM files.

### SUMMARY

In an aspect of the present disclosure, a method for digital imaging and communications in medicine (DICOM) file processing is provided. The method may include receiving a request for processing a DICOM file. The DICOM file may include data of metadata and pixel data. The method may also include parsing at least part of the metadata of the DICOM file. The method may further include writing the data of the DICOM file to one or more data streams based on the parsed metadata.

In some embodiments, the writing data of the DICOM file to one or more data streams based on the parsed metadata may include obtaining a size of the pixel data of the DICOM file from the parsed metadata; and writing the data of the DICOM file to the one or more data streams based on whether the size of the pixel data exceeds a threshold.

In some embodiments, the threshold may be determined at least based on the size of cache of a system executing the method.

In some embodiments, the writing the data of the DICOM file to the one or more data streams based on whether the size of the pixel data exceeds a threshold may include in response to determining that the size of the pixel data exceeds the threshold, writing the parsed metadata of the DICOM file and the pixel data of the DICOM file to one of the one or more data streams. At least part of the pixel data of the DICOM file may be not parsed.

In some embodiments, the writing the data of the DICOM file to the one or more data streams based on whether the size of the pixel data exceeds a threshold may include in response to determining that the size of the pixel data does not exceed the threshold, parsing the pixel data of the DICOM file; and writing the parsed metadata of the DICOM file and the parsed pixel data of the DICOM file to one of the one or more data streams.

In some embodiments, the method may further include segmenting the pixel data of the DICOM file into a plurality of pixel groups; and generating a plurality of DICOM sub-files based on the plurality of pixel groups respectively and the DICOM file.

In some embodiments, the method may further include writing data of the plurality of DICOM sub-files to the one or more data streams.

In some embodiments, the method may further include transferring the plurality of DICOM sub-files or the one or more data streams in a parallel fashion.

In some embodiments, the segmenting the pixel data into a plurality of pixel groups may include obtaining an optimal size for segmentation; and segmenting the pixel data into the plurality of pixel groups based on the optimal size.

In some embodiments, the optimal size may be adjustable in real time.

In some embodiments, each of the plurality of pixel groups may carry a sequence number that indicates a segmentation order of the pixel group in the pixel data.

In another aspect of the present disclosure, a method for digital imaging and communications in medicine (DICOM) file processing is provided. The method may include receiving a request for processing a DICOM file. The method may also include obtaining a first data stream including data of the DICOM file. The method may also include parsing metadata of the DICOM file from the first data stream. The method may further include writing the parsed metadata of the DICOM file and pixel data of the DICOM file to a second data stream. The pixel data may be not parsed.

In some embodiments, the second data stream may be a file stream, and the processing a DICOM file may include storing the DICOM file in a local file system.

In some embodiments, the second data stream may be a network stream, and the processing a DICOM file may include transmitting the DICOM file to an external system.

In some embodiments, the metadata of the DICOM file may include a recorded data length of the pixel data of the DICOM file, and the method may further include determining an actual data length of the pixel data of the DICOM file; and obtaining the pixel data from the first data stream based on whether the recorded data length is equal to the actual data length.

In another aspect of the present disclosure, a method for digital imaging and communications in medicine (DICOM)file processing is provided. The method may include obtaining a DICOM file. The method may also include obtaining pixel data of the DICOM file. The method may also include segmenting the pixel data into a plurality of pixel groups. The method may further include generating, based on the plurality of pixel groups and the DICOM file, a plurality of DICOM sub-files.

In some embodiments, the method may further include transferring the plurality of DICOM sub-files in a parallel fashion.

In some embodiments, the generating, based on the plurality of pixel groups and the DICOM file, a plurality of DICOM sub-files may include parsing metadata of the DICOM file; and for each of the plurality of pixel groups, generating a DICOM sub-file corresponding to the pixel group by using, according to a DICOM protocol, the parsed metadata of the DICOM file and the pixel group.

In some embodiments, the generating, based on the plurality of groups and the DICOM file, a plurality of DICOM sub-files may include for each of the plurality of pixel groups, determining a DICOM sub-file corresponding to the pixel sub-data by using the pixel group as pixel data of the DICOM sub-file with the pixel sub-data.

In some embodiments, the segmenting the parsed pixel data into a plurality of pixel groups may include obtaining an optimal size for segmentation; and segmenting the parsed pixel data into the plurality of pixel groups based on the optimal size.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary file transmission system according to some embodiments of the present disclosure;

FIG. 2 is a schematic diagram illustrating hardware and/or software components of an exemplary computing device according to some embodiments of the present disclosure;

FIG. 3 is a schematic diagram illustrating hardware and/or software components of an exemplary mobile device according to some embodiments of the present disclosure;

FIGS. 4A-4D are block diagrams illustrating exemplary processing devices according to some embodiments of the present disclosure;

FIG. 5 is a flowchart illustrating an exemplary process for DICOM file processing according to some embodiments of the present disclosure;

FIG. 6 is a schematic diagram illustrating an exemplary process for DICOM file processing according to some embodiments of the present disclosure;

FIG. 7 is a flowchart illustrating an exemplary process for DICOM file processing according to some embodiments of the present disclosure;

FIG. 8 is a flowchart illustrating an exemplary process for DICOM file processing according to some embodiments of the present disclosure;

FIG. 9 is a flowchart illustrating an exemplary process for DICOM file storage according to some embodiments of the present disclosure;

FIG. 10 is a flowchart illustrating an exemplary process for DICOM file forwarding according to some embodiments of the present disclosure;

FIG. 11 is a schematic diagram illustrating an exemplary process for DICOM file transmission according to some embodiments of the present disclosure;

FIG. 12 is a schematic diagram illustrating an exemplary process for generating a plurality of DICOM sub-files according to some embodiments of the present disclosure;

FIG. 13 is a schematic diagram illustrating an exemplary process for generating a plurality of DICOM sub-files according to some embodiments of the present disclosure;

FIG. 14 is a schematic diagram illustrating an exemplary process for DICOM file storage according to some embodiments of the present disclosure; and

FIG. 15 is a schematic diagram illustrating an exemplary process for generating an initial DICOM file according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in descending order. However, the terms may be displaced by another expression if they achieve the same purpose.

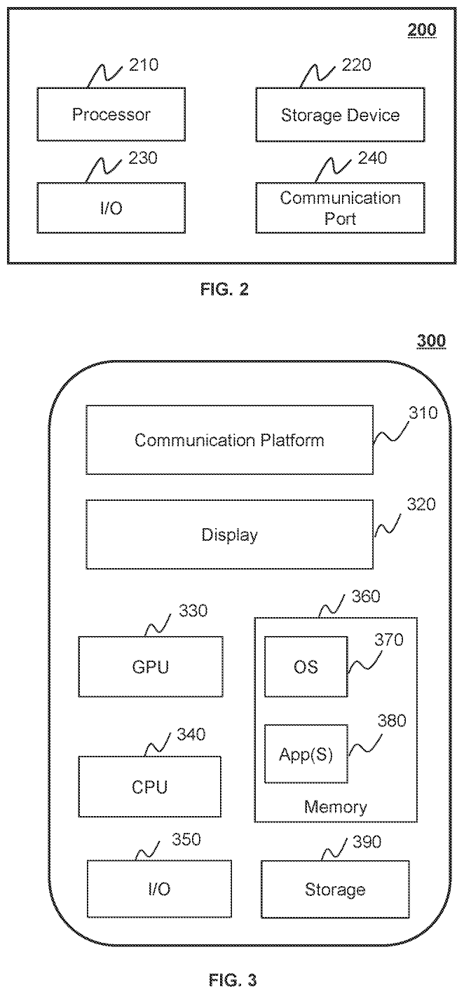

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processor 210 as illustrated in FIG. 2) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “image data” in the present disclosure is used to collectively refer to raw data (e.g., projection data) and/or images of various forms, including two-dimensional (2D) image data, three-dimensional (3D) image data, four-dimensional (4D) image data, etc. The terms “pixel” in the present disclosure are used to refer to an element of an image. The subject may include a biological subject (e.g., a human, an animal), a non-biological subject (e.g., a phantom), etc. For example, the subject may include a specific part, organ, and/or tissue of a patient. As another example, the subject may include the head, the brain, the neck, the breast, the heart, the lung, the stomach, blood vessels, soft tissues, or the like, or any combination thereof. The term “object” or “subject” are used interchangeably in the present disclosure. The term “modality” used herein broadly refers to an imaging or treatment method or technology that gathers, generates, processes, and/or analyzes imaging information of a subject or treatments the subject. The terms “plurality of” in the present disclosure may refer to that a count (number) of a related object is more than two (i.e., at least two). In the present disclosure, the terms “flow” and “stream” are used interchangeably.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The flowcharts used in the present disclosure illustrate operations that systems implement according to some embodiments of the present disclosure. It is to be expressly understood the operations of the flowcharts may be implemented not in order. Conversely, the operations may be implemented in an inverted order, or simultaneously. Moreover, one or more other operations may be added to the flowcharts. One or more operations may be removed from the flowcharts.

As used in the present disclosure, the DICOM protocol is an international standard for medical images and related information. The DICOM protocol defines a medical image format whose quality satisfies the clinical needs and can be used for data exchange. The DICOM protocol supports/provides a C-Store service. The C-Store service includes a C-Store SCP (service class provider) and a C-Store SCU (service class user). The C-Store SCP is a service end that supports DICOM file reception. The C-Store SCU is a client end that supports sending DICOM files. For archiving or storing a DICOM file, the C-Store SCP generally parses data of the DICOM file received from network stream(s) through the memory of the C-Store SCP, and then writes the parsed data from the memory to a disk or outgoing network stream(s). In such cases, the memory usage of the service end may surge with the size of the received DICOM file, which may lead to the collapse of the service end if too many such files are processed. For sending a DICOM file, the C-Store SCU may read data of the DICOM file through a memory of the C-Store SCU, and then write the data from the memory to network stream(s). In such cases, memory usage of the client end may surge with the size of the DICOM file, and cause system failure or unacceptable performances.

Generally, in the above scenarios, as the size of the DICOM file becomes larger, it is necessary to continuously improve the hardware configuration to ensure the normal operation of the system (e.g., the PACS), which increases hardware costs. In addition, a lot of unused data of the DICOM file may be parsed, resulting in the overall performance of data storage and the subsequent data processing being low. Therefore, it is desirable to provide systems and methods for file processing, which can effectively solve the above problems and improve file transmission efficiency and/or performance.

An aspect of the present disclosure relates to systems and/or methods for DICOM file processing. The systems may obtain a request for processing a DICOM file. The systems may parse at least part of metadata of the DICOM file. The systems may further write data of the DICOM file to one or more data streams based on the parsed metadata. At least part of the data of the DICOM file written to the data stream(s) is not parsed. For example, the systems may write the parsed metadata and pixel data of the DICOM file to the data stream(s), and some of the pixel data is not parsed.

According to some embodiments of the present disclosure, when a local file system (e.g., the PACS) receives the DICOM file, only part of the DICOM file (e.g., the metadata of the DICOM file) may be parsed and written to the memory of the PACS, and remaining part of the DICOM file (e.g., the pixel data of the DICOM file) may not be parsed and not be written to the memory of the PACS. For example, the remaining part of the DICOM file may be directly written to the data stream(s) for storing in a disk of the PACS without through the memory. Generally, the amount of the metadata of the DICOM file is small (e.g., less than 16 Kb) while the amount of the pixel data of the DICOM file is large (e.g., greater than 500 M). Sometimes, it is not necessary to parse all data of the DICOM file to the memory or cache of the PACS, therefore reducing the memory consumption, maintaining stable usage of the memory, and improving the system performance.

Another aspect of the present disclosure relates to systems and/or methods for DICOM file processing. The systems may obtain a DICOM file. The systems may obtain pixel data of the DICOM file. The systems may segment the pixel data of the DICOM file into a plurality of pixel groups. The systems may further generate, based on the plurality of pixel groups and the DICOM file (e.g., metadata of the DICOM file), a plurality of DICOM sub-files. Further, the systems may write data of the plurality of DICOM sub-files to one or more data streams for further processing.

Some existing technologies may perform physical division on the DICOM file for improving transmission performance. However, the DICOM file after division may not have complete label information (e.g., metadata) of the DICOM file. In such cases, a client end or a server end that receives a division of the DICOM file cannot obtain the label information from the division, resulting in that medical images in the DICOM file cannot be displayed until all divisions of the DICOM file are received and combined. According to some embodiments of the present disclosure, the DICOM file may be divided to generate a plurality of DICOM sub-files. Each of the DICOM sub-files may include complete label information (e.g., the metadata) of the DICOM file and support the DICOM protocol. Accordingly, the plurality of DICOM sub-files can be transferred in a parallel fashion according to the DICOM protocol, thereby helping the client end or the server end to normally display the transmitted medical images during transmission.

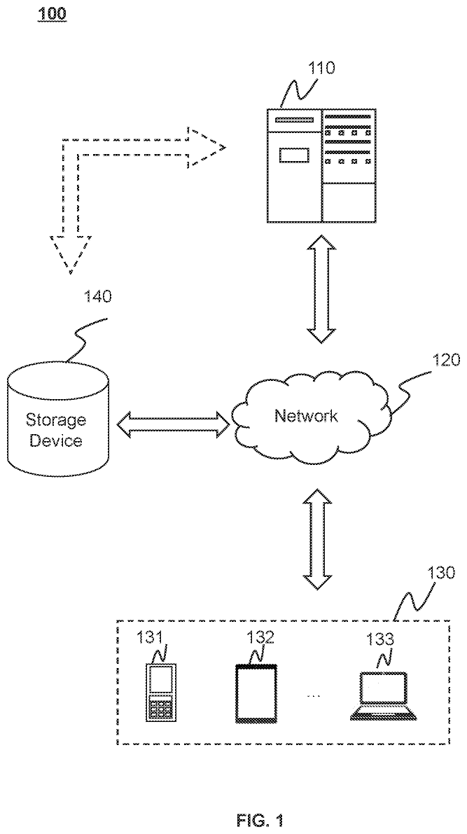

FIG. 1 is a schematic diagram illustrating an exemplary file transmission system according to some embodiments of the present disclosure. In some embodiments, the file transmission system 100 may be configured to achieve transmission of DICOM files. As illustrated in FIG. 1, the file transmission system 100 may include a processing device 110, the network 120, terminal(s) 130, and a storage device 140.

The processing device 110 may process data and/or information. The data and/or information may be obtained from the terminal(s) 130 and/or the storage device 140. In some embodiments, the processing device 110 may obtain a request for processing a DICOM file, e.g., from the terminal 130. The request may be data stream(s) including how to process the DICOM file and the DICOM file. The processing device 110 may parse at least part of metadata of the DICOM file. The processing device 110 may write data of the DICOM file to one or more data streams based on the parsed metadata. At least part of the data of the DICOM file written to the data stream(s) may be not parsed. For example, the processing device 110 may write the parsed metadata and pixel data of the DICOM file (that is not parsed) to the data stream(s) for storing and/or forwarding. As another example, the processing device 110 may parse the pixel data of the DICOM file. The processing device 110 may segment the pixel data of the DICOM file into a plurality of pixel groups. The processing device 110 may generate, based on the plurality of pixel groups and the DICOM file, a plurality of DICOM sub-files. The processing device 110 may transfer the plurality of DICOM sub-files in a parallel fashion according to a DICOM protocol. For instance, the processing device 110 may transfer the plurality of DICOM sub-files by writing data of the plurality of DICOM sub-files to the one or more data streams.

In some embodiments, the DICOM file may include image data of a subject, e.g., medical image data acquired by a medical imaging device. For example, after the medical imaging device performs a scan of the subject or the portion thereof, the medical imaging device may generate the image data of the subject or the portion thereof. The image data of the subject or the portion thereof may need to be transmitted to an image file system (e.g., a local file system such as the PACS) for processing (e.g., storing, transmitting, and/or forwarding). The image data and/or reports of the subject in a form of an electronic file may be transmitted digitally from/to the image file system, such that a user (e.g., an authorized user such as a doctor, a third party, etc.) can retrieve the image data for diagnosis and/or research purposes from or send the image data to the image file system via the terminal 130. For instance, the image file system may include a picture archiving and communication system (PACS). The PACS may be a medical imaging technology configured to provide storage and access to image data from multiple modalities. In some embodiments, the processing device 110 may be part of the image file system (e.g., the PACS). In some embodiments, the image data may be transmitted to/from the image file system according to a preset transmission protocol (e.g., a DICOM protocol).

In some embodiments, the processing device 110 may be a single server or a server group. The server group may be centralized or distributed. In some embodiments, the processing device 110 may be local or remote. For example, the processing device 110 may access information and/or data stored in the terminal(s)130, and/or the storage device 140 via the network 120. As another example, the processing device 110 may be directly connected to the terminal(s) 130, and/or the storage device 140 to access stored information and/or data. In some embodiments, the processing device 110 may be implemented on a cloud platform. For example, a cloud platform may include a private cloud, a public cloud, a hybrid cloud, a community cloud, a distributed cloud, an inter-cloud, and a multi-cloud, or the like, or any combination thereof. In some embodiments, the processing device 110 may be implemented by a computing device 200 having one or more components as illustrated in FIG. 2 or be a portion of the terminal 130.

The network 120 may include any suitable network that can facilitate the exchange of information and/or data of the file processing system. In some embodiments, one or more components of the file processing system (e.g., the terminal 130, the processing device 110, the storage device 140, etc.) may communicate information and/or data with one or more components of the file processing system 100 via the network 120. The network 120 may include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (“VPN”), a satellite network, a telephone network, routers, hubs, server computers, or the like, or a combination thereof. For example, the network 120 may include a wireline network, an optical fiber network, a telecommunication network, a local area network, a wireless local area network (WLAN), a metropolitan area network (MAN), a public telephone switched network (PSTN), a Bluetooth™ network, a ZigBee™ network, a near field communication (NFC) network, or the like, or a combination thereof. In some embodiments, the network 120 may include one or more network access points. For example, the network 120 may include wired and/or wireless network access points, such as base stations and/or Internet exchange points, through which one or more components of the file processing system may be connected to the network 120 to exchange data and/or information.

The terminal 130 may input/output signals, data, information, etc. In some embodiments, the terminal 130 may enable user interaction with the processing device 110. In some embodiments, the terminal 130 (e.g., a mobile device 131, a tablet computer 132, a laptop computer 133, etc.) may communicate with the processing device 110 via the network 120. For example, the terminal 130 may send a request for processing a DICOM file according to a user instruction to the processing device 110. As another example, a user may access the image file system for obtaining a DICOM file via the terminal 130. For instance, the terminal 130 may obtain user input information (e.g., a search query or a file transmission request) through an input device (e.g., a keyboard, a touch screen, a brain wave monitoring device), and transmit the input information to the processing device 110 for obtaining the DICOM file stored in the image file system. As still another example, the terminal 130 may generate a plurality of DICOM sub-files based on a DICOM file. The terminal 130 may transmit the plurality of DICOM sub-files to the processing device 110 for further processing. In some embodiments, the terminal 130 may display image data in a DICOM file on a display device (e.g., a screen of the terminal 130).

The storage device 140 may store data (e.g., image data of a subject, a DICOM file), instructions, and/or any other information. In some embodiments, the storage device 140 may store data obtained from the terminal(s) 130 and/or the processing device 110. For example, the storage device 140 may store data of the DICOM file received by the processing device 110. In some embodiments, the storage device 140 may store data and/or instructions executed or used by the processing device 110 to perform exemplary methods described in the present disclosure. In some embodiments, the storage device 140 may include a mass storage device, a removable storage device, a volatile read-write memory, a read-only memory (ROM), or the like, or any combination thereof. In some embodiments, the storage device 140 may be a part of the image file system. For example, the storage device 140 may include a memory (e.g., a volatile read-write memory) as the memory of the image file system. The memory of the image file system may store the metadata of the received DICOM file. As another example, the storage device 140 may include a mass storage device or a removable storage device as the disk of the image file system. The disk of the image file system may store data of the received DICOM file (e.g., the metadata and the pixel data of the received DICOM file.

In some embodiments, the storage device 140 may be connected to the network 120 to communicate with one or more components (e.g., the processing device 110, the terminal 130, etc.) of the file transmission system. One or more components of the file transmission system may access the data or instructions in the storage device 140 via the network 120. In some embodiments, the storage device 140 may be a part of the processing device 110. Alternatively, the storage device 140 may be independent and directly or indirectly connected to the processing device 110. For example, when the storage device 140 includes the memory and the disk of the image file system. The memory may be a part of the processing device 110, and the disk may be directly or indirectly connected to the processing device 110 (e.g., via the network 120).

In some embodiments, both the processing device 110 and the storage device 140 may be parts of the image file system (e.g., a local file system such as the PACS). In such cases, the image file system may also be referred to as a server end, and the terminal(s) may also be referred to as a client end. The server end and the client end may achieve communication (e.g., file transmission) via the network 120.

It should be noted that the above description regarding the file transmission system is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the file transmission system may include one or more additional components and/or one or more components of the file transmission system described above may be omitted. In some embodiments, a component of the file transmission system may be implemented on two or more sub-components. Two or more components of the file transmission system may be integrated into a single component.

FIG. 2 is a schematic diagram illustrating exemplary hardware and/or software components of an exemplary computing device according to some embodiments of the present disclosure. The computing device 200 may be configured to implement any component of the file transmission system. For example, the terminal 130, the processing device 110, and/or the storage device 140 may be implemented on the computing device 200. Although only one such computing device is shown for convenience, the computer functions relating to the file transmission system as described herein may be implemented in a distributed fashion on a number of similar platforms, to distribute the processing load. As illustrated in FIG. 2, the computing device 200 may include a processor 210, a storage device 220, an input/output (I/O) 230, and a communication port 240.

The processor 210 may execute computer instructions (e.g., program codes) and perform functions of the processing device 110 in accordance with techniques described herein. The computer instructions may include, for example, routines, programs, objects, components, signals, data structures, procedures, modules, and functions, which perform particular functions described herein. In some embodiments, the processor 210 may perform instructions obtained from the terminal 130 and/or the storage device 140. In some embodiments, the processor 210 may include one or more hardware processors, such as a microcontroller, a microprocessor, a reduced instruction set computer (RISC), an application-specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field-programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be noted that the computing device 200 in the present disclosure may also include multiple processors. Thus operations and/or method steps that are performed by one processor as described in the present disclosure may also be jointly or separately performed by the multiple processors. For example, if in the present disclosure the processor of the computing device 200 executes both operation A and operation B, it should be understood that operation A and operation B may also be performed by two or more different processors jointly or separately in the computing device 200 (e.g., a first processor executes operation A and a second processor executes operation B, or the first and second processors jointly execute operations A and B).

The storage device 220 may store data/information obtained from the terminal 130, the storage device 140, or any other component of the file transmission system. In some embodiments, the storage device 220 may include a mass storage device, a removable storage device, a volatile read-and-write memory, a read-only memory (ROM), or the like, or any combination thereof. In some embodiments, the storage device 220 may store one or more programs and/or instructions to perform exemplary methods described in the present disclosure.

The I/O 230 may input or output signals, data, and/or information. In some embodiments, the I/O 230 may enable user interaction with the processing device 110. In some embodiments, the I/O 230 may include an input device and an output device. Exemplary input devices may include a keyboard, a mouse, a touch screen, a microphone, a camera capturing gestures, or the like, or a combination thereof. Exemplary output devices may include a display device, a loudspeaker, a printer, a projector, a 3D hologram, a light, a warning light, or the like, or a combination thereof. Exemplary display devices may include a liquid crystal display (LCD), a light-emitting diode (LED)-based display, a flat panel display, a curved screen, a television device, a cathode ray tube (CRT), or the like, or a combination thereof.

The communication port 240 may be connected with a network (e.g., the network 120) to facilitate data communications. The communication port 240 may establish connections between the processing device 110 and the terminal 130, or the storage device 140. The connection may be a wired connection, a wireless connection, or a combination of both that enables data transmission and reception. The wired connection may include an electrical cable, an optical cable, a telephone wire, or the like, or any combination thereof. The wireless connection may include a Bluetooth™ network, a Wi-Fi network, a WiMax network, a WLAN, a ZigBee™ network, a mobile network (e.g., 3G, 4G, 5G), or the like, or any combination thereof. In some embodiments, the communication port 240 may be a standardized communication port, such as RS232, RS485, etc. In some embodiments, the communication port 240 may be a specially designed communication port. For example, the communication port 240 may be designed in accordance with the digital imaging and communications in medicine (DICOM) protocol.

FIG. 3 is a schematic diagram illustrating exemplary hardware and/or software components of an exemplary mobile device according to some embodiments of the present disclosure. In some embodiments, the processing device 110 or the terminal 130 may be implemented on the mobile device 300.

As illustrated in FIG. 3, the mobile device 300 may include a communication module 310, a display 320, a graphics processing unit (GPU) 330, a central processing unit (CPU) 340, an I/O 350, a memory 360, and storage 390. The CPU 340 may include interface circuits and processing circuits similar to the processor 210. In some embodiments, any other suitable component, including but not limited to a system bus or a controller (not shown), may also be included in the mobile device 300. In some embodiments, a mobile operating system 370 (e.g., iOS™, Android™, Windows Phone™) and one or more applications 380 may be loaded into the memory 360 from the storage 390 in order to be executed by the CPU 340. The applications 380 may include a browser or any other suitable mobile apps for receiving and rendering information relating to imaging from the mobile device 300. User interactions with the information stream may be achieved via the I/O devices 350 and provided to the processing device 110 and/or other components of the file transmission system 100 via the network 120.

To implement various modules, units, and their functionalities described in the present disclosure, computer hardware platforms may be used as the hardware platform(s) for one or more of the elements described herein. A computer with user interface elements may be used to implement a personal computer (PC) or any other type of workstation or terminal device. A computer may also act as a server if appropriately programmed.

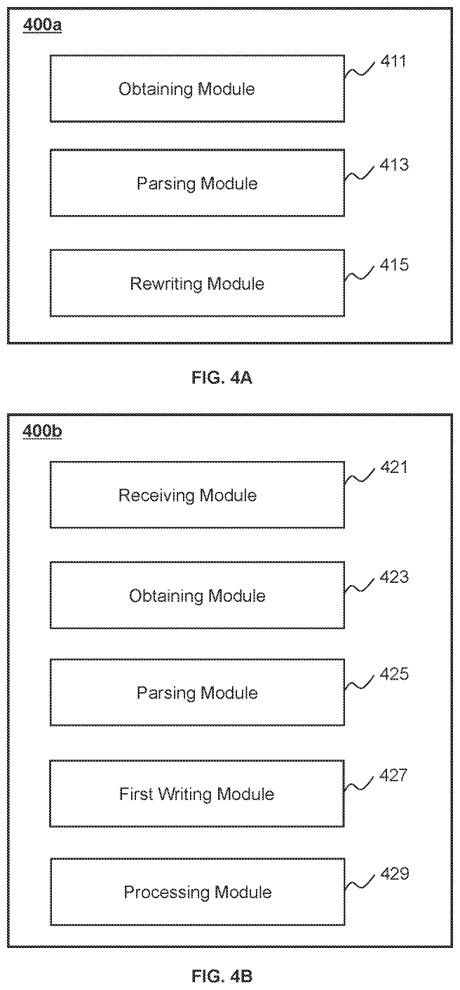

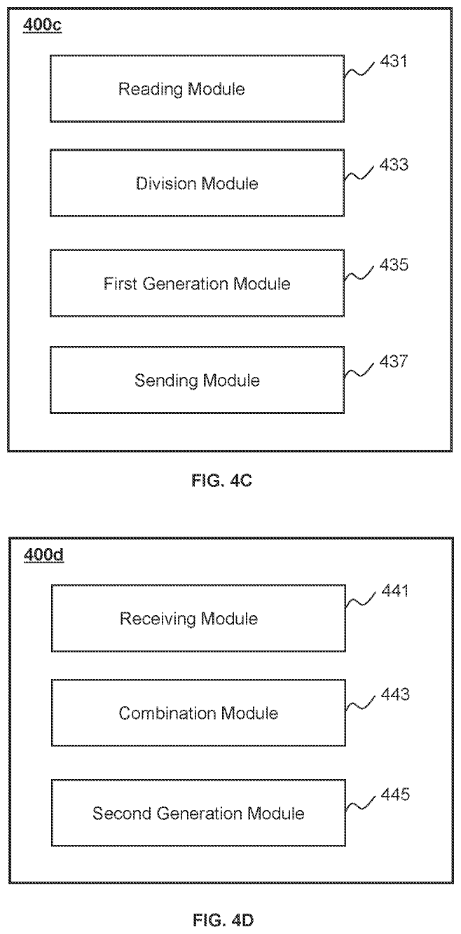

FIGS. 4A-4D are schematic diagrams illustrating exemplary processing devices according to some embodiments of the present disclosure.

As shown in FIG. 4A, the processing device 400a may include an obtaining module 411, a parsing module 413, and a rewriting module 415.

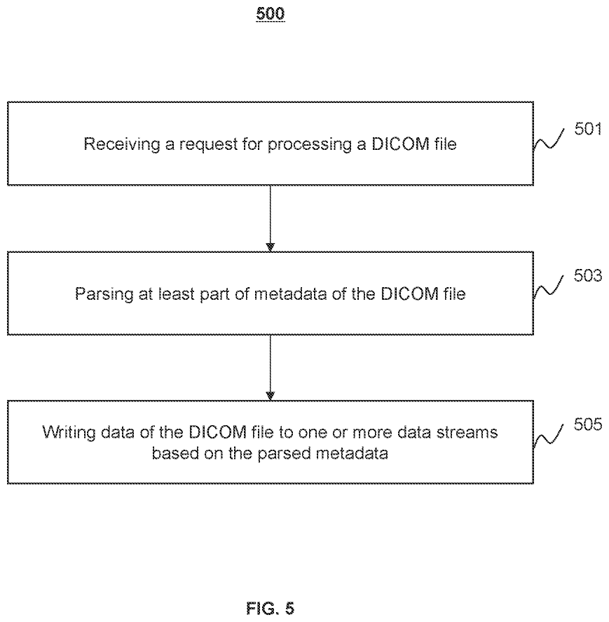

The obtaining module 411 may be configured to obtain image/information from one or more components of the file transmission system 100. For example, the obtaining module 411 may receive a request for processing a DICOM file. The request may be carried by a data stream. The data stream may include instructions about how to process the DICOM file and/or data of the DICOM file. More descriptions regarding receiving the request may be found elsewhere in the present disclosure (e.g., operation 501 in FIG. 5 and relevant descriptions thereof.

The parsing module 413 may be configured to parse information related to the request (e.g., the information and/or data of the data stream which carries the request). For example, the parsing module 413 may parse the instruction about how to process the DICOM file. As another example, the parsing module 413 may parse metadata of the DICOM file from the data stream. As still another example, the parsing module 413 may parse pixel data of the DICOM file from the data stream. More descriptions regarding the parsing operation may be found elsewhere in the present disclosure (e.g., operations 503 and 505 and relevant descriptions thereof).

The rewriting module 415 may be configured to write data of the DICOM file to one or more data streams. For example, the rewriting module 415 may write the parsed metadata of the DICOM file to the data stream(s). As another example, the rewriting module 415 may write parsed pixel data and/or unparsed pixel data to the data stream(s). More descriptions regarding the writing operation may be found elsewhere in the present disclosure (e.g., operation 505 and relevant descriptions thereof).

As shown in FIG. 4B, the processing device 400b may include a receiving module 421, an obtaining module 423, a parsing module 425, a first writing module 427, and a processing module 429.

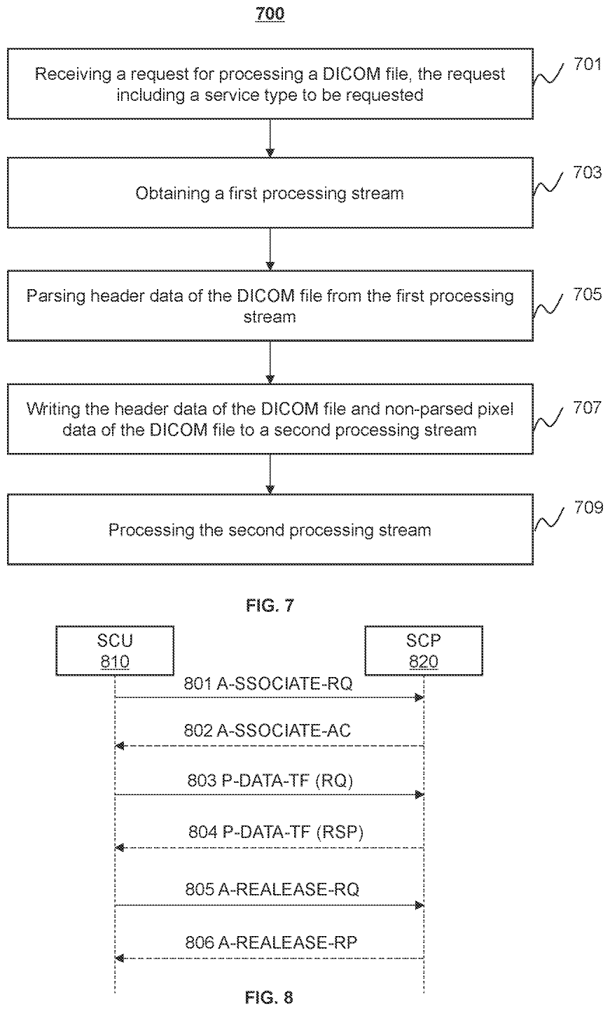

The receiving module 421 may be configured to receive a request for processing a DICOM file, e.g., from a client end (e.g., the terminal 130). The request may include a service type (e.g., a kind of server that is requested). The service type may include a storage service, a forwarding service, a query service, or the like, or any combination thereof. More descriptions regarding the request may be found elsewhere in the present disclosure (e.g., operation 701 in FIG. 7 and relevant descriptions thereof).

The obtaining module 423 may be configured to obtain a first processing stream. More descriptions regarding the first processing stream may be found elsewhere in the present disclosure (e.g., operation 703 in FIG. 7 and relevant descriptions thereof).

The parsing module 425 may be configured to parse the header data in the first processing stream. The header data may include a recorded size (e.g., a recorded data length) of the unparsed medical image data. More descriptions regarding the parsing operation may be found elsewhere in the present disclosure (e.g., operation 705 in FIG. 7 and relevant descriptions thereof).

The first writing module 427 may be configured to write the parsed header data into a second processing stream. The second processing stream may contain unparsed medical image data in the first processing stream. Data sources of the unparsed medical image data written to the second processing stream may be from the first processing stream or be locally resourced. More descriptions regarding the writing operation may be found elsewhere in the present disclosure (e.g., operation 707 in FIG. 7 and relevant descriptions thereof).

The processing module 429 may be configured to process the second processing stream. For example, the processing module 429 may send the second processing stream externally (e.g., to a real destination of the DICOM file) according to an instruction of the column storage database. More descriptions regarding the processing of the second processing stream may be found elsewhere in the present disclosure (e.g., operation 707 in FIG. 7 and relevant descriptions thereof).

In some embodiments, when the service type is a storage service, the first processing stream may be a network stream sent by the terminal 130, and the second processing stream may be a local file stream. In such cases, the processing device 400b may further include a first determination module (not shown) and a second writing module (not shown). The first determination module may be configured to determine an actual data length of the unparsed medical image data and determine whether the actual data length of the unparsed medical image data is consistent with (e.g., equal to) the recorded data length of the unparsed medical image data. The second writing module may be configured to obtain the unparsed medical image data from the first processing stream when the recorded data length is consistent with (e.g., equal to) the actual data length, and write the unparsed medical image data into the second processing stream.

In some embodiments, when the service type is the forwarding service, the first processing stream may be a network stream sent by the terminal 130, and the second processing stream may be a network stream forwarding externally. In such cases, the processing device 400b may further include a third writing module (not shown) configured to write the medical image data stored locally into the second processing stream when there is locally stored medical image data that is the same as the unparsed medical image data. When there is no locally stored medical image data that is the same as the unparsed medical image data, the third writing module may obtain the unparsed medical image data from the first processing stream and write the unparsed medical image data into the second processing stream.

As shown in FIG. 4C, the processing device 400c may include a reading module 431, a division module 433, a first generation module 435, and a sending module 437.

The reading module 431 may be configured to obtain initial pixel data in an initial DICOM file. More descriptions regarding the initial pixel data may be found elsewhere in the present disclosure (e.g., operation 1101 in FIG. 11 and the relevant descriptions thereof).

The division module 432 may be configured to divide the pixel data into a plurality of pixel groups. More descriptions regarding the division operation may be found elsewhere in the present disclosure (e.g., operation 1103 in FIG. 11 and the relevant descriptions thereof).

The first generation module 433 may be configured to generate a plurality of DICOM sub-files based on the plurality of pixel groups and the initial DICOM file. More descriptions regarding the generation of the DICOM sub-files may be found elsewhere in the present disclosure (e.g., operation 1105 in FIG. 11 and the relevant descriptions thereof).

The sending module 434 may be configured to send the plurality of DICOM sub-files according to a preset transmission protocol. More descriptions regarding the sending operation may be found elsewhere in the present disclosure (e.g., operation 1107 and FIG. 11 and the relevant descriptions thereof).

As shown in FIG. 4D, the processing device 400d may include a receiving module 441, a combination module 443, and a second generation module 445.

The receiving module 441 may be configured to receive a plurality of DICOM sub-files and read pixel data of each of the plurality of DICOM sub-files. More description regarding the plurality of DICOM sub-files and the pixel data may be found elsewhere in the present disclosure (e.g., operation 1401 in FIG. 14) and relevant descriptions thereof).

The combining module 443 may be configured to determine target pixel data by merging/combining a plurality of pixel data of the plurality of DICOM sub-files. More descriptions regarding the determination of the target pixel data may be found elsewhere in the present disclosure (e.g., operation 1403 in FIG. 14 and relevant descriptions thereof).

The second generation module 445 may be configured to generate an initial DICOM file based on the target pixel data and one of the plurality of DICOM sub-files. More descriptions regarding the generation of the initial DICOM file may be found elsewhere in the present disclosure (e.g., operation 1405 in FIG. 14 and relevant descriptions thereof).

In some embodiments, the above modules may be different modules in a processing device, or may be a module that implements functions of two or more modules mentioned above. As illustrated above, processing devices 400a, 400b, and 400d may be exemplary configurations of the processing device 110. In some embodiments, the processing devices 400a, 400b, and 400d may share one or more of the modules illustrated above. For instance, the processing devices 400a, 400b, and 400d may be part of a same system and share a same module. For example, the obtaining module 411, the receiving module 421, the obtaining module 423, and/or the receiving module 441 may be a same module. In some embodiments, the processing devices 400a-400d may be different devices belonging to different parties. For example, the processing device 400a, the processing device 400b, and/or the processing device 400d may belong to a server end (e.g., the C-Store SCP of the image file system). As another example, the processing device 400c may belong to the server end (e.g., the C-Store SCU) or a client end (e.g., the terminal 130).

FIG. 5 is a flowchart illustrating an exemplary process for DICOM file processing according to some embodiments of the present disclosure. In some embodiments, the process 500 may be implemented as a set of instructions (e.g., an application) stored in a storage device (e.g., the storage device 140, the storage device 220, and/or the storage 390). The processing device 400a (e.g., the processor 210, the CPU 340, and/or one or more modules illustrated in FIG. 4A) may execute the set of instructions, and when executing the instructions, the processing device 400a may be configured to perform the process 500. The operations of the illustrated process presented below are intended to be illustrative. In some embodiments, the process 500 may be accomplished with one or more additional operations not described and/or without one or more of the operations discussed. Additionally, the order of the operations of process 500 illustrated in FIG. 5 and described below is not intended to be limiting. In some embodiments, operations of the process 500 are described in connection with receiving a DICOM file from a client end (e.g., the terminal 130). It should be noted that operations of the process 500 may be applied to send a DICOM file as well. In some embodiments, operations of the process 500 may be implemented by a server end such as an image file system (e.g., a local file system such as the PACS or the C-Store SCU of the local file system) of the file transmission system. For example, the processing device 400a may be a part of the image file system. Alternatively, operations of the process 500 may be implemented by a client end (e.g., the terminal 130) of the file transmission system or an external system (e.g., an external file system) that can communicate with the file transmission system 100 and supports the DICOM protocol.

In 501, the processing device 400a (e.g., the obtaining module 411) may receive a request for processing a DICOM file.

In some embodiments, the DICOM file may store medical image data of an object (e.g., a patient) that is acquired from a scan of the object using an imaging device (e.g., the medical imaging device) in a DICOM format. The DICOM file may include metadata (also referred to as header or header data), pixel data (i.e., the medical image data), etc. The metadata of the DICOM file may include identification information relating to the medical image data and/or the object. The metadata may be organized as a standard series of tags. The series of tags may further be organized into groups of data elements. Different groups may correspond to different identifiers (IDs) and information. For example, a group with ID “0010” may contain patient information, e.g., the patient's name, the patient's identification number, the patient's birth date, etc. which correspond to different tags (i.e., with different tag identifiers (IDs)). As another example, a group with ID “0018” may contain acquisition information (e.g., acquisition parameters that are used to acquire the medical image data). As still another example, a group with ID “0028” may contain an image presentation and be responsible for the display of the medical image data. As a further example, a group with ID “0002” may contain file meta information (e.g., a transfer syntax, an abstract syntax, unique identifier (UID) information, etc.) of the DICOM file. The metadata may be followed by the pixel data. The pixel data may correspond to a single attribute/tag with tag ID “7FE0” that contains the pixel data corresponding to the image data. The pixel data may be stored as a series of 0s and 1s. In some embodiments, the pixel data in the DICOM file may begin at the tag with ID (7FE0, 0010). In some embodiments, for brevity, tags corresponding to the metadata may also be referred to as meta tags, and tags corresponding to the pixel data may also be referred to as pixel tags.

In some embodiments, the processing device 400a may receive the request via/from one or more data stream (e.g., a network stream such as a socked network stream) (also referred to as first data stream(s)) that carries the request. The data stream may include instructions about how to process the DICOM file (e.g., what kind of service is requested) and/or data (e.g., the metadata, the pixel data, etc.) of the DICOM file. Taking the request and the data of the DICOM file included in a first data stream as an example, the first data stream may include multiple segments that are arranged/transmitted in sequence. Each of the multiple segments may have a limited size (e.g., a limited data length), which depends on the type of the first data stream. The multiple segments may include a first segment and one or more other segments arranged following the first segment. The first segment of the multiple segments may include a command set and a part of data (e.g., the metadata and/or a part of the pixel data) of the DICOM file. The command set may include instructions about how to process the DICOM file (e.g., to store, forward, etc., the DICOM file) and/or other meta information (e.g., the metadata or the file meta information) of the DICOM file. Following segments after the first segment of the multiple segments may include other parts of the DICOM file. For example, the pixel data may be divided into a plurality of segments according to a specific data length, and the following segments may include a section of the plurality of segments. More descriptions regarding the first data stream may be found elsewhere in the following disclosure (e.g., FIGS. 7-9 and relevant descriptions thereof).

In 503, the processing device 400a (e.g., the parsing module 413) may parse at least part of the metadata of the DICOM file.

In some embodiments, the processing device 400a may parse the at least part of the metadata of the DICOM file from the first data stream (e.g., from one or more segments of the first data stream). In some embodiments, the at least part of the metadata may include information regarding the size (e.g., the data length) of the pixel data of the DICOM file. The processing device 400a may parse the size (e.g., the data length) of the pixel data of the DICOM file from the first data stream. In some embodiments, the processing device 400a may parse information in the first data stream in sequence until the tag of (7FE0, 0010) of the DICOM file is detected. That is, the processing device 400a may parse all information (e.g., the metadata of the DICOM file) before the pixel data of the DICOM file in the first data stream. For example, the processing device 400a may parse all information before the tag of (7FE0, 0010) and parse information of 12 bytes which is subsequently after the tag of (7FE0, 0010) and describes the pixel data, and may not parse subsequent information after the information of 12 bytes. More descriptions regarding parsing the at least part of the metadata of the DICOM file may be found elsewhere in the following disclosure (e.g., FIGS. 7-9 and relevant descriptions thereof).

In 505, the processing device 400a (e.g., the parsing module 413, the rewriting module 415, etc.) may write data of the DICOM file to one or more data streams (also referred to as second data stream(s)) based on the parsed metadata.

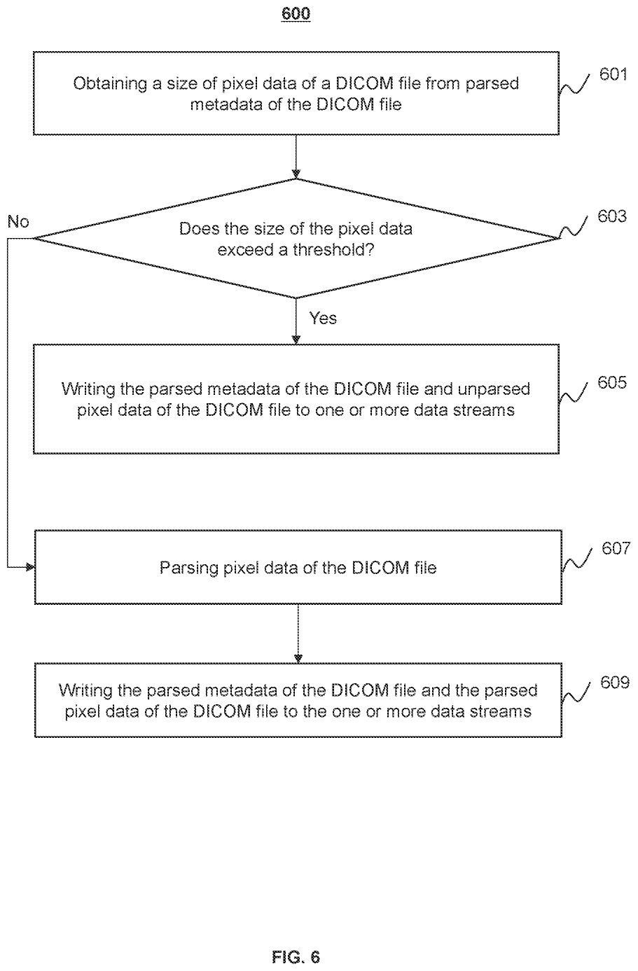

In some embodiments, at least part of the data of the DICOM file written to the second data stream may be not parsed. For example, the processing device 400a may obtain the size (e.g., the data length) of the pixel data from the parsed metadata. The processing device 400a may write the data of the DICOM file to the second data stream based on whether the size (e.g., the data length) of the pixel data exceeds a threshold (e.g., a threshold length). For instance, in response to determining that the data length of the pixel data does not exceed the threshold length, the processing device 400a may parse the pixel data of the DICOM file. The processing device 400a may write the parsed metadata of the DICOM file and the parsed pixel data of the DICOM file to the second data stream. In response to determining that the data length of the pixel data exceeds the threshold length, the processing device 400a may not parse the pixel data of the DICOM file and directly write the parsed metadata of the DICOM file and the unparsed pixel data of the DICOM file to the second data stream(s). Alternatively, in response to determining that the data length of the pixel data exceeds the threshold length, the processing device 400a may parse the pixel data of the DICOM file. The processing device 400a may write the parsed metadata and the parsed pixel data to the second data stream(s). More descriptions regarding writing the data of the DICOM file to the second data stream(s) based on the parsed metadata may be found elsewhere in the following disclosure (e.g., FIG. 6 and relevant descriptions thereof).

In some embodiments, according to different types of how to process the DICOM file, the second data stream may have different types. For example, if the DICOM file is requested to be stored in the image file system (e.g., a local file system), the second data stream may include a file stream, such that the data of the DICOM file written to the second data stream can be stored in a storage device (e.g., the storage 140 or a disk such as a hard disk) of the local file system. As another example, if the DICOM file is requested to be forwarded to an external system via the image file system, the second data stream may include a network stream (e.g., a socket network stream), such that the data of the DICM written to the second data stream can be transferred or forwarded to the external system via a network (e.g., the network 120). More descriptions regarding the different types of the second data stream may be found elsewhere in the following disclosure (e.g., FIGS. 7-10 and relevant descriptions thereof).

In some embodiments, before operation 505, the processing device 400a may determine whether the DICOM file is a DICOM sub-file. As used herein, a DICOM sub-file refers to a file in a DICOM format that is generated based on a part of pixel data of a specific DICOM file. Such DICOM sub-file may include a private tag in metadata of the DICOM sub-file for labeling the DICOM sub-file, e.g., indicating the type of the DICOM sub-file, a sequence number of the DICOM sub-file in its corresponding specific DICOM file, a total count (or number) of DICOM sub-files corresponding to the specific DICOM file, the name of the DICOM sub-file, the size (e.g., the data length) of the DICOM sub-file, or the like, or any combination thereof. For example, the processing device 400a may determine whether the DICOM file carried by the first data stream is a DICOM sub-file based on the parsed metadata of the DICOM file. In response to determining that the DICOM file carried by the first data stream is a DICOM sub-file, the processing device 400a may create a document folder in the disk of the image file system and store data of the DICOM sub-files corresponding to the specific DICOM file in the document folder until the last DICOM sub-file of the DICOM sub-files corresponding to the specific DICOM file is received. In some embodiments, the processing device 400a may restore the specific DICOM file by combining the DICOM sub-files based on sequence numbers of the DICOM sub-files stored in the document folder.

In some embodiments, different from operation 501, the processing device 400a may directly obtain/receive the DICOM file according to the DICOM protocol. The processing device 400a may obtain the pixel data of the DICOM file. The processing device 400a may segment the pixel data of the DICOM file to a plurality of pixel groups, more descriptions of which may be similar to the segmenting parsed pixel data to a plurality of pixel groups as described in operation 608 in FIG. 6. The processing device 400a may generate a plurality of DICOM sub-files based on the plurality of pixel groups respectively, more descriptions of which may be found elsewhere in the present disclosure (e.g., FIGS. 11-15 and relevant descriptions thereof). Each of the plurality of DICOM sub-files may satisfy the DICOM standard and in the DICOM format. Further, the processing device 400a may transfer the plurality of DICOM sub-files in a parallel fashion. For instance, the processing device 400a may write data of the plurality of DICOM sub-files to one or more data streams (e.g., the second data stream(s)). The processing device 400a may transfer the one or more second data streams in a parallel fashion for achieving transferring the DICOM file. Merely by way of example, data of each of the plurality of DICOM sub-files may be written to one of the second data stream(s). The second data streams(s) each of which carries the data of each of the plurality of DICOM sub-files may be transferred in a parallel fashion. Alternatively, the plurality of DICOM sub-files may be transferred according to the DICOM protocol in a parallel fashion. In some embodiments, before segmenting the pixel data of the DICOM file, the processing device 400a may determine a size (e.g., a data length) of the pixel data based on the metadata of the DICOM file. The processing device 400a may determine whether the size (e.g., the data length) exceeds a threshold (e.g., a threshold length). In response to determining that the data length of the pixel data exceeds the threshold length, the processing device 400a may perform the segmentation operation on the pixel data of the DICOM file. More descriptions regarding the determining whether the size (e.g., the data length) of the pixel data exceeds the threshold (e.g., the threshold length) may be similar to that described in operation 603 in FIG. 6, which is not repeated herein.

It should be noted that the above description regarding the process 500 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added to or omitted from the process 500. For example, the process 500 may include an additional operation for causing the image file system to archive the DICOM file. As another example, a storing operation may be added elsewhere in the process 500. In the storing operation, the processing device 400a may store information and/or data used or obtained disclosed elsewhere in the present disclosure. In some embodiments, an operation of the process 500 may be divided into at least two sub-operations. For example, operation 505 may be divided into two sub-operations, one of which is for determining whether the data length of the pixel data of the DICOM file exceeds the threshold length and another one of which is for writing the data of the DICOM file based on the determination results. In some embodiments, the request for processing the DICOM file may include only how to process the DICOM file without the DICOM file, and the DICOM file may be pre-stored in the image file system. For example, the processing device 400a may receive a request for querying and/or retrieving the DICOM file from the image file system. The request may include the file meta information contained in the group “0002” of the DICOM file to be queried and/or retrieved. The processing device 400a may parse the request to obtain the file meta information of the DICOM file. The processing device 400a may determine where the DICOM file is stored and write data of the DICOM file from the image file system (e.g., from a file stream containing the data of the DICOM file) to the second data stream (e.g., a network data stream such as a socked stream). The processing device 400a may transmit the DICOM file to a client end (e.g., the terminal 130) that requests to query and/or retrieve the DICOM file for further processing.

FIG. 6 is a flowchart illustrating an exemplary process for DICOM file processing according to some embodiments of the present disclosure. In some embodiments, the process 600 may be implemented as a set of instructions (e.g., an application) stored in a storage device (e.g., the storage device 140, the storage device 220, and/or the storage 390). The processing device 110 (e.g., the processor 210, the CPU 340, and/or one or more modules illustrated in FIG. 4A) may execute the set of instructions, and when executing the instructions, the processing device 400a may be configured to perform the process 600. The operations of the illustrated process presented below are intended to be illustrative. In some embodiments, the process 600 may be accomplished with one or more additional operations not described and/or without one or more of the operations discussed. Additionally, the order of the operations of process 600 illustrated in FIG. 6 and described below is not intended to be limiting. In some embodiments, operation 505 in FIG. 5 may be achieved by the process 600.

In 601, the processing device 400a (e.g., the rewriting module 415) may obtain a size (e.g., a data length) of pixel data of a DICOM file from parsed metadata of the DICOM file.

As described in connection with operations 501 and 503, the metadata of the DICOM file may store the size (e.g., the data length) of the pixel data of the DICOM file. The processing device 400a may directly obtain the size (e.g., the data length) of the pixel data of the DICOM file after parsing at least part of the metadata of the DICOM file from the first data stream.

In 603, the processing device 400a (e.g., the rewriting module 415) may determine whether the size (e.g., the data length) of the pixel data exceeds a threshold (also referred to as a first threshold) (e.g., a first threshold length).

In some embodiments, in response to determining that the size (e.g., the data length) of the pixel data exceeds the first threshold (e.g., the first threshold length), the process 500 may proceed to operation 605. In response to determining that the data length of the pixel data does not exceed the threshold length, the process 500 may proceed to operation 607.

In some embodiments, the first threshold (e.g., the first threshold length) may be determined at least based on the size of cache of the system (e.g., the image file system) executing the process 500 and/or 600. For example, the image file system (e.g., a local file system such as the PACS) may have a cache (or buffer) of a certain size. The first threshold may be determined to be equal to or less than the certain size of the cache, such that parsed pixel data of the DICOM file can be temporarily stored in a buffer and then be transferred to the disk (e.g., a hardware disk) of the image file system. In some embodiments, the first threshold may be determined based on a read-write speed of the disk of the image file system and/or a transfer speed of the first data stream that carries the DICOM file, as well as the size of cache. For example, the greater the size of cache, the greater the first threshold may be determined. As another example, the faster the read-write speed of the disk is, the greater the first threshold may be determined. As another example, if the read-write speed of the disk is slower than the transfer speed of the first data stream, the greater a difference between the read-write speed of the disk and the transfer speed of the first data stream, the less the first threshold may be determined. In some embodiments, the first threshold may be determined based on statistics of historical data received by the image file system. For example, the first threshold may be determined based on user experience regarding the historical data. As another example, the first threshold may be determined by machine learning based on historical data. For instance, a machine learning model may be trained based on the historical data. An input of the machine learning model may include parameters of the data length of the pixel data of the DICOM file, the read-write speed of the disk, the transfer speed of the first data stream, the size of cache, etc., and an output of the machine learning model may be the first threshold. In some embodiments, the first threshold may be adjustable in real time.

In 605, in response to determining that the data length of the pixel data exceeds the threshold (i.e., the first threshold), the processing device 400a (e.g., the rewriting module 415) may write the parsed metadata of the DICOM file and pixel data of the DICOM file to one or more data streams (i.e., the second data stream(s) described in FIG. 5).

In some embodiments, the processing device 400a may directly obtain the pixel data of the DICOM file from the first data stream without parsing, i.e., the pixel data of the DICOM file is not parsed. The processing device 400a may write the parsed metadata and the unparsed pixel data to the second data stream.

In some embodiments, the processing device 400a may parse a part of the pixel data of the DICOM file and write the parsed pixel data and unparsed pixel data of the DICOM file to the second data stream. For instance, in response to determining that the size (e.g., the data length which is denoted by D) of the pixel data exceeds the first threshold (e.g., the first threshold length), the processing device 400a may parse the part of the pixel data from the first data stream based on a second threshold (e.g., a second threshold length). For example, the second threshold (e.g., the second threshold length) may be a threshold with a fixed data length, which may be determined based on the size of memory of the image file system that receives the DICOM file. The processing device 400a may parse a part of the pixel data of the DICOM file a data length of which is equal to the second threshold length and do not parse remaining pixel data of the DICOM file in the first data stream. As another example, the second threshold length may be a dynamic threshold that is equal to a certain percentage (e.g., denoted by P such as 1%, 5%, 10%, 20%, etc.) multiplying the data length of the pixel data of the DICOM file. The processing device 400a may parse a part of the pixel data of the DICOM file a data length of which is equal to the certain percentage multiplying the data length of the pixel data of the DICOM file (i.e., A*D) and not parse remaining pixel data of the DICOM file a data length of which is equal to (1−A)*D.

In 607, in response to determining that the data length of the pixel data does not exceed the threshold length, the processing device 400a (e.g., the rewriting module 415) may parse the pixel data of the DICOM file. In some embodiments, the processing device 400a may parse the pixel data of the DICOM file by parsing information from and after the tag with an ID of (7FE0, 0010) in the first data stream(s).

In 608, the processing device 400a (e.g., the rewriting module 415) may write the parsed metadata of the DICOM file and the parsed pixel data of the DICOM file to the one or more data streams (i.e., the second data stream(s)).

In some embodiments, the processing device 110 may directly write the parsed metadata of the DICOM file and the parsed pixel data of the DICOM file to the second data stream. Alternatively, the processing device 400a may segment the parsed pixel data into a plurality of pixel groups. Each of the plurality of pixel groups may carry a sequence number that indicates a segmentation order of the pixel group in the parsed pixel data. For example, the processing device 400a may obtain an optimal size (e.g., an optimal data length) for segmentation. The processing device 110 may segment the parsed pixel data into the plurality of pixel groups based on the optimal size (e.g., the optimal data length). For instance, a data length of one of the plurality of pixel groups may be equal to the optimal data length. In some embodiments, the optimal size (e.g., the optimal data length) may be adjustable in real time or a default setting of the PACS (e.g., which is determined based on user experiences). For example, the optimal size (e.g., the optimal data length) may be determined based on the size of ache or buffer of the PACS. As another example, the optimal size (e.g., the optimal data length) may be determined based on the transfer speed of the second data stream(s). Further, the processing device 400a may write the plurality of pixel groups to the second data stream(s). For example, the processing device 400a may write one or more of the plurality of pixel groups to one of the second data stream(s). The processing device 400a may transfer the one or more second data stream(s) in a parallel fashion.

It should be noted that the above description regarding the process 600 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added to or omitted from the process 600. In some embodiments, an operation of the process 600 may be divided into two or more sub-operations. In 608, for the plurality of pixel groups, the processing device 400a may not generate the plurality of DICOM streams. The processing device 400a may directly store the plurality of pixel groups in ache or buffer of the PACS and then transfer the plurality of pixel groups from ache to the second data stream.

FIG. 7 is a schematic diagram illustrating an exemplary process for DICOM file processing according to some embodiments of the present disclosure. In some embodiments, the process 700 may be implemented as a set of instructions (e.g., an application) stored in a storage device (e.g., the storage device 140, the storage device 220, and/or the storage 390). The processing device 400b (e.g., the processor 210, the CPU 340, and/or one or more modules illustrated in FIG. 4B) may execute the set of instructions, and when executing the instructions, the processing device 400b may be configured to perform the process 700. The operations of the illustrated process presented below are intended to be illustrative. In some embodiments, the process 700 may be accomplished with one or more additional operations not described and/or without one or more of the operations discussed. Additionally, the order of the operations of process 700 as illustrated in FIG. 7 and described below is not intended to be limiting. In some embodiments, the process 700 may be implemented by the processing device 400a. Alternatively, the process 700 may be implemented by a server end such as an image file system (e.g., a local file system such as the PACS or the C-Store SCP thereof) of the file transmission system 100. For example, the processing device 400b may be a part of the server end (e.g., the image file system).

In 701, the processing device 400b (e.g., the receiving module 421) may receive a request for processing a DICOM file. The request may include a service type that is to be requested.

In some embodiments, the request may be sent by a client end (also referred to as an opposite end) (e.g., the terminal 130). The client end may send the request in a format of a processing stream (e.g., a network stream such as a socket stream). The processing stream may include a command set and data sets that are generated based on a DICOM protocol format. The data sets may be the first processing stream in 703. According to the command set, the processing device 400b may obtain the service type of the request for further providing corresponding processing (e.g., the subsequent parsing processing). The service type may include a plurality of types according to actual needs, e.g., a querying service, a storage service, an obtaining service, a sending/forwarding service, a deletion service, etc., which is not limited herein.

In some embodiments, before operation 701, the client end and the server end (e.g., the processing device 400b) may establish a network connection. The present disclosure may not limit the mode of how to establish the network connection. For example, the processing device 400b may receive a request for establishing a network connection from the client end. The processing device 400b may determine a service type that the client end is requested according to the request for establishing a network connection. The processing device 400b may determine whether the service type is supported locally (e.g., whether the service type is supported by the server client). In response to determining that the service type is supported locally, the processing device 120b may feed a confirmation massage back to the client end and establish the network connection with the client end. It should be noted that in response to determining that the service type is not supported locally, the processing device 120b may feed a rejection message back to the client end and not establish the network connection with the client end. In some embodiments, the processing device 400b may be configured with different operating environments in advance to support different service types.

In 703, the processing device 400b (e.g., the obtaining module 423) may obtain a first processing stream (also referred to as a first processing flow). The first processing stream may be the same as or similar to the first data stream(s) as described in operation 501 in FIG. 5.

In some embodiments, the first processing stream may be sent by the client end. The first processing stream may include medical image data (e.g., pixel data) of the DICOM file.

In 705, the processing device 400b (e.g., the parsing module 425) may parse header data of the DICOM file from the first processing stream. The header data of the DICOM file may be the same as or similar to the metadata of the DICOM file as described in operation 501 in FIG. 5.

In some embodiments, the processing device 400b may parse the header data in the first processing stream based on the format specification of the DICOM protocol. The header data and medical image data in the first processing stream may be distinguished based on an end mark(e.g., a tag ID), so that the header data in the first processing stream may be parsed based on the end mark. For example, the tag ID of (7FE0,0010) may be used as an end mark. Data before the end mark may be the header data, and data after the end mark may be the medical image data. That is, the header data is followed by the medical image data. During an actual implementation process, the processing device 400b may parse data from the beginning of the first processing stream until the end mark. In some embodiments, the processing device 110 may store the parsed header data in a memory (e.g., a memory of the server end (e.g., a memory of the image file system)). In some embodiments, the header data may usually be 12 bytes and mainly includes file meta information of the DICOM file (i.e., meta tags). The meta tag may define transfer syntax, record a data length of the medical image data in the DICOM file, etc.

In 707, the processing device 400b (e.g., the first writing module 427) may write the parsed header data and unparsed pixel data of the DICOM file to a second processing stream. The second processing stream may be the same as or similar to the second data stream(s) as described in operation 503 in FIG. 5.

In some embodiments, as described in operation 705, the parsed header data may be stored in the memory of the server end. The processing device 400b may write the parsed header data from the memory to the second processing stream. At the same time, the processing device 400b may write the unparsed medical image data to the second processing stream. In some embodiments, data sources of the unparsed medical image data written to the second processing stream may be from the first processing stream or be locally sourced, which is not limited herein. For example, the processing device 400b may write the unparsed medical image data to the second processing stream from the first processing stream or a local storage device (e.g., a disk or the memory of the image file system).

In some embodiments, the type of the second processing stream and the mode for processing the second processing stream (e.g., how to process the second processing stream) may correspond to the service type of the request. For example, the service type may be the storage service. Correspondingly, the first processing stream may be a network stream sent by the client end, and the second processing stream may be a local file stream. The application scenario of the storage service is mainly that the client end sends a DICOM file to the server end, and the server end receives, stores, and/or achieves the DICOM file. For example, the client end that supports the DICOM protocol may send the DICOM file to the server end through the C-Store. In such cases, the first processing stream may be the network stream sent by the client end. For example, the network stream may include a socket network stream. The present disclosure may not limit a specific transmission protocol used by the network stream. The second processing stream may be the local file stream, via which a local placement of the DICOM file can be achieved (e.g., the DICOM file is stored in the disk locally).

For illustration purposes, an exemplary process for achieving a storage service may be illustrated in FIG. 8. The process in FIG. 8 may include operations 801-806 and involve two implementation subjects (e.g., a client end (e.g., SCU) 810 and a server end (e.g., SCP) 820).

In 801, the client end 810 may send a request of A-SSOCIATE-RQ for establishing a network connection to the server end 820. Since there may be various service requests, only when the server end 820 supports the service type, the server end 820 may feed a confirmation message of A-SSOCIATE-AC back to the client end 810. In such cases, a network connection may be established between the server end 820 and the client end 810. If the service type requested by the client end 810 is not supported by the server end 820, the server end 820 may feed a rejection message of A-SSOCIATE-RJ back to the client end 810.

In 802, the client end 810 may receive the A-SSOCIATE-AC returned by the server end 820, which indicates that the network connection has been established between the client end 810 and the server end 820.

In 803, the client end 810 may send a processing stream of P-DATA-TF (RQ) to the server end 820. The P-DATA-TF (RQ) may carry a command set and data sets. For example, the P-DATA-TF (RQ) may be the socket network stream. The server end 820 may monitor and receive the socket network stream through a socket port. It should be noted that the socket network stream may be composed of sections of data, that is, various segments. Each of the various segments may have a limited maximum data length. In the socket network stream, the command set may be followed by the data sets. A first segment of the various segments may include the command set and a part of the data sets, and following segments of the various segments may include remaining of the data sets.

In some embodiments, the client end 810 may send the processing stream of P-Data-TF (RQ) to the server end 820 according to an instruction of the column storage database. The column storage database may include the C-Store, which is not limited herein. The C-Store is a relational database designed for a quick query. To achieve a quicker query performance, the C-Store stores data according to columns. Different columns in the same table may be stored in different projections, and the projections may have overlapping parts. The projection used herein refers to a set of data storage columns.

In some embodiments, after receiving the P-Data-TF (RQ), the server end 820 may parse the command set in the P-Data-TF (RQ) to determine the service type required by the client end 810, so as to use/initiate a specific service to process the data sets carried subsequently in the P-Data-TF (e.g., to process the first processing stream). In related technologies, the processing mode may need to write all the received data sets to the memory, which may result in too much consumption of memory resources. Alternatively, the data may be directly written into the local cache, and retrieved from the cache when subsequent use, which may result in a sharp increase of I/O (Input/Output) operations, thereby reducing system processing performance.

To avoid or reduce the above two situations, the present disclosure mainly analyzes, based on the DICOM standard for the DICOM file format, features of data elements and their components in the data sets of the DICOM file, so as to perform de-memory and de-cache processing on the medical image data. That is, the present disclosure may bypass the memory and/or cache, and directly establish a pipeline connection with the real destination (e.g., a target receiving end) of the DICOM file, thereby achieving quick and efficient processing of the medical image data.

In some embodiments, the server end 820 may obtain data sets in the P-Data-TF (RQ) (i.e., the data part (the first processing stream) in the socket network stream) to parse the header data in the first processing stream. It should be noted that the server end 820 in FIG. 8 provides the storage service. In the actual implementation process, the server end 820 may monitor obtained network data (i.e., the socket network stream) to dynamically obtain data needed. For example, the server end 820 may obtain the command set and the first processing stream by adopting the dynamic obtaining mode. As the obtaining of the network data is an I/O operation, a blocking I/O mechanism and a non-blocking I/O mechanism may be adopted to obtain the network data. Further, a synchronous I/O mechanism and an asynchronous I/O mechanism may be adopted to obtain the network data.

As used herein, the concepts of blocking I/O and non-blocking I/O are program-levels, which are used to describe the problem that after the program requests to operate system I/O operation, if the I/O resource is not prepared, what should the program do. For example, if the I/O resource is not prepared, the blocking I/O mechanism may wait, while the non-blocking I/O mechanism may keep performing, that is, the thread would always be inquired until the I/O resource is ready.

As used herein, the concepts of synchronous I/O and asynchronous I/O are operating system levels, which are used to describe the problem that, after the operating system receives the program request for the I/O operation, if the I/O resource is not prepared, how to respond to the program. For example, if the I/O resource is not prepared, the synchronous I/O may not respond until the I/O resource is ready, while the asynchronous I/O may feed a tag back to mark a return target after the event that the I/O resource is ready. Through the different I/O mechanisms, the server end 820 may parse the first processing stream section by section. For example, only the header data in the first processing stream may be parsed. The header data may usually be 12 bytes and not exceed 16 Kb. In some embodiments, the header data may be cut out from the first processing stream through an end mark (e.g., the tag ID (7fe0,0010)), and the parsed header data may be recorded and saved.

In some embodiments, the server end 820 may write the parsed header data to the second processing stream, that is, to the local file stream of the server end 820. At the same time, the unparsed medical image data in the first processing stream may be written into the second processing stream. Finally, the DICOM file may be placed/stored locally through the second processing stream. At this point, the storage service provided by the server end 820 may end.

In 804, the server end 820 may selectively feed the P-Data-TF (RSP) back to the client end 810 to indicate the end of the storage service as a response.

In 805, after the client end 810 receives the P-Data-TF (RSP), the client end 810 may send a request of A-Release-RQ for releasing the network connection to the server end 820. In 806, after receiving the A-Release-RQ, the server end 820 may feed A-Release-RP back to the client end 810 to indicate the confirmation of releasing the network connection with the client end 810.

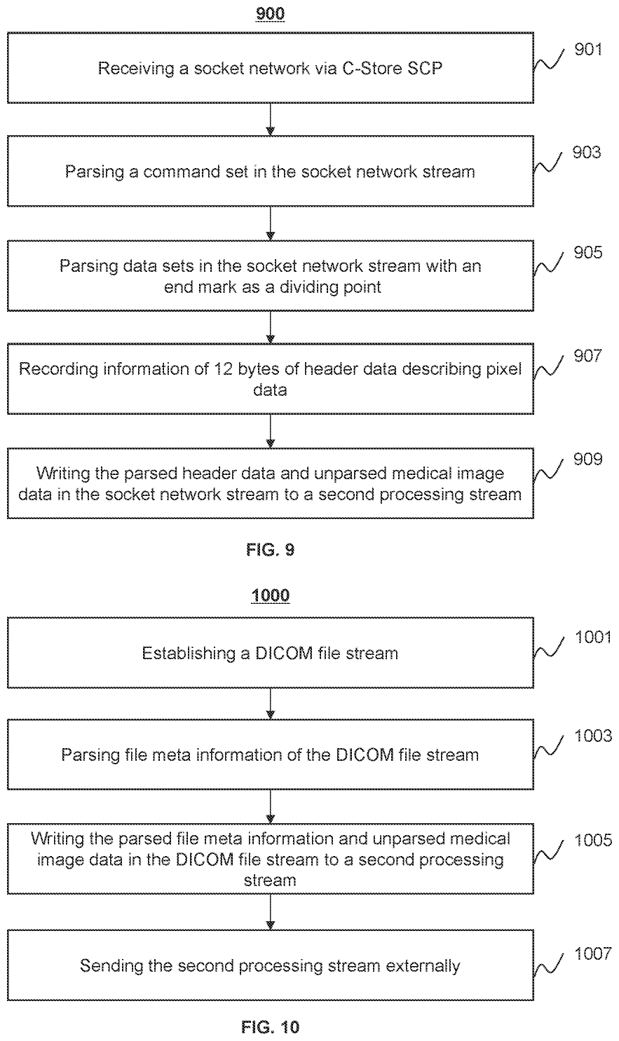

The above descriptions may be the exemplary process when the service type is the storage service, the brief process of which may be illustrated in FIG. 9. The process of 900 may include operations 901-909 as follows, which may be implemented by the processing device 400b or the server end (e.g., the server end 820).

In 901, a socket network stream may be received through C-Store SCP.

In 903, a command set may be parsed in the socket network stream.

In 905, data sets in the socket network may be parsed with an end mark as a dividing point.

In 907, information of 12 bytes of header data describing pixel data (e.g., header data describing medical image data in the socket network stream) may be recorded.

In 909, the parsed header data and unparsed medical image data in the socket network stream may be written to a second processing stream.

According to the process 700, 800, or 900 of the present disclosure, there is no need to write all received data sets into the memory of the image file system, thereby avoiding or reducing the excessive consumption of memory resources. At the same time, there is no need to write all received data sets into the local cache, and then retrieve them from the local cache, thereby avoiding the sharp increase of I/O operations that reduces the system processing performance.

In some embodiments, the header data may include the recorded data length of the unparsed medical image data. Correspondingly, before processing the second processing stream, the processing device 400b may determine an actual data length of the unparsed medical image data. If the recorded data length is consistent with the actual data length, the processing device 400b may obtain the unparsed medical image data from the first processing stream, and write the unparsed medical image data to the second processing stream.

As described elsewhere in the present disclosure, the header data and the medical image data in the first processing stream are mainly distinguished by the end mark. In the embodiments, the processing device 400b may start statistics from the end mark until the end of the medical image data in the first processing stream, so as to determine the actual data length of the unparsed medical image data. The end of the medical image data in the first processing stream may be a preset end mark (e.g., a preset character or a preset fixed byte), which is not limited herein.

When the service type is the storage service, the data source of the unparsed medical image data may be the first processing stream, so that the unparsed medical image data may need to be obtained from the first processing stream. In such cases, the recorded data length may be compared with the actual data length. If the recorded data length is inconsistent with the actual data length, the unparsed medical image data may not be obtained from the first processing stream. In such cases, a resend message may be fed back to the client end (e.g., the client end 810), so that the client end may resend the first processing stream. If the recorded data length is consistent with the actual data length, the unparsed medical image data may be obtained from the first processing stream, and the unparsed medical image data may be written to the second processing stream without passing the memory or the local cache of the image file system.

According to some embodiments of the present disclosure, by determining the actual data length of the unparsed medical image data, if the recorded data length is consistent with the actual data length, the processing device 120b may obtain the unparsed medical image data from the first processing stream, and write the unparsed medical image to the second processing stream. The determination result of whether the actual data length and the recorded data length of the medical image data are consistent (or equivalent) may be used to indicate whether there is an error in the medical image data, thereby avoiding the situation of writing wrong medical image data to the second processing stream, and ensuring the accuracy of the data in the subsequent local placement.

In some embodiments, the service type may be a forwarding service. Correspondingly, the first processing stream may be a network stream sent by the client end (e.g., the client end 810), and the second processing stream may be a network stream that is forwarded externally (e.g., forwarded to the client end).

An application scenario of the forwarding service may be that a client end (e.g., the client end 810) sends the DICOM file to a server (e.g., the server end 820) and the server end may forward the DICOM file to other client ends or other server ends. In some embodiments, the client end that supports the DICOM protocol may send the DICOM file to the server end through C-Store. In such cases, the first processing stream refers to the network stream sent by the client end to the server end. The network stream may include a socket network stream. The present disclosure may not limit the transmission protocol used by the network stream. The second processing stream may be a network stream that the server end forwarded externally, through which the DICOM file may be forwarded to other client ends or server ends. In such cases, there is no need to write all received data sets to the memory of the image file system, so as to avoid excessive consumption of the memory resource. Alternatively, there is no need to write all received data sets into the local cache of the image file system, and then retrieve them from the local cache, thereby avoiding the sharp increase of I/O operations which may reduce the system processing performance.

In some embodiments, before processing the second processing stream, if there is locally stored medical image data that is the same as the unparsed medical image data, the processing device 400b may write the locally stored medical image data to the second processing stream. Alternatively, if there is no locally stored medical image data that is the same as the unparsed medical image data, the processing device 11b may obtain the unparsed medical image data from the first processing stream and write the unparsed medical image data to the second processing stream.

It can be seen from the above embodiments that when the service type is the forwarding service, that is, the terminal end sends the DICOM file to the server end, and the server end forwards the DICOM file to other client ends or server ends, the server end may not pre-store the DICOM file in advance or have stored the DICOM file. Therefore, different processes may be provided for different situations. Whether there is locally stored medical image data that is the same as the unparsed medical image data may be determined based on the parsed header data. For example, the parsed header data may include a unique identifier used to correspond with the medical image data, such as tag information (e.g., one or more tags), including an image width and height, a data transmission format, a patient name, a patient birthday, a medical record hospital, a medical record department, the description of a disease, etc. For a certain medical image data, to determine whether the medical image data has been stored locally, whether the tag information of the medical image data has been stored locally may be searched. If the tag information of the medical image data has been stored locally, the medical image data may be indicated to have been stored locally. If the tag information of the medical image data has not been stored locally, the medical image data may be indicated to be not stored locally.

To facilitate understanding, the implementation process of the forwarding service may also be described in connection with FIG. 8.

In 801, the client end 810 may send a network connection request A-SSOCIATE-RQ to the server end 820. Since there may be various service types requested, only when the service type is supported by the server end 820, the server end 820 may selectively feed a confirmation message of A-SSOCIATE-AC back to the client end 810. In such cases, a network connection may be established between the server end 820 and the client end 810. If the service type is not supported by the server end 820, the server end 820 may feed a rejection message of A-SSOCIATE-RJ back to the client end 810.

In 802, the client end 810 may receive the A-SSOCIATE-AC returned by the server end 820, which indicates the network connection has been established between the client end 810 and the server.

In 803, the client end 810 may send a processing stream of P-Data-TF (RQ) that carries a command set and data sets to the server end 820. The P-Data-TF (RQ) may be a socket network stream. The server end 820 may monitor through a socket port to receive the socked network stream. It should be noted that the socket network stream may be composed of sections of data, that is, various segments. Each of the various segments may have a maximum data length. In the socket network stream, the command set may be followed by the data sets. A first segment of the various segments may include the command set and a part of the data sets, and the following segments of the various segments may include the remaining of the data sets. In some embodiments, the client end 810 may send the P-Data-TF (RQ) to the server end 820 according to an instruction of C-Store.

In some embodiments, after receiving the P-Data-TF (RQ), the server end 820 may parse the command set in the P-Data-TF (RQ) to determine the service type required by the client end 810, so as to use/initiate a specific service to process the data sets carried by subsequently in the P-Data-TF (i.e., to process the first processing stream). In related technologies, the processing mode may include writing all the received data sets to the memory, which may result in too much consumption of memory resources. Alternatively, the data may be directly written into the local cache, and retrieved from the cache when subsequent use, which may result in a sharp increase of I/O operations, thereby reducing system processing performance.

To avoid or reduce the above two situations, the present disclosure mainly analyzes, based on the DICOM standard for the DICOM file format, the features of data elements and their components in the data sets of the DICOM file, so as to perform de-memory and de-cache processing on the medical image data. That is, the present disclosure may bypass the memory and/or cache, and directly establish a pipeline connection with the real destination of the DICOM file, thereby achieving quick and efficient processing of the medical image data.

In some embodiments, the data sets in P-Data-TF (RQ) (i.e., the data part of the socket network stream) may be the first processing stream. As the unparsed medical image data in the first processing stream may have been stored or not stored, the server end 820 may determine whether there is locally stored medical image data that is the same as the unparsed medical image data based on the parsed header data.

In some embodiments, the medical image data may be verified by a verification code, and each medical image data may correspond to a unique verification code (e.g., a UID (such as an SOP Instance UID) of the DICOM file corresponding to the medical image data). Different DICOM files corresponding to different medical image data may correspond to different SOP instance UIDs. The present disclosure may not limit the modes of how to determine whether there is locally stored medical image data that is the same as the unparsed medical image data based on the parsed header data. For example, the server end 820 may obtain the verification code corresponding to the unparsed medical image data. The server end 820 may compare the verification code corresponding to the unparsed medical image data with a verification code corresponding to locally stored medical image data. If there is the same verification code as the verification code corresponding to the unparsed medical image data, the server end 820 may determine that there is locally stored medical image data that is the same as the unparsed medical image data. If there is no same verification code as the verification code corresponding to the unparsed medical image data, the server end 820 may determine there is no locally stored medical image data that is the same as the unparsed medical image data.

If the server end 820 determines that there is locally stored medical image data that is the same as the unparsed medical image data, the server end 820 may write the parsed header data and the locally stored medical image data which is the same as unparsed medical image data to the second processing stream (i.e., the network stream that is forwarded externally). If the server end 820 determines that there is no locally stored medical image data that is the same as the unparsed medical image data, the server end 820 may write the parsed header data and the unparsed medical image data in the first processing stream to the second processing stream (i.e., the network stream that is forwarded externally). Accordingly, the DICOM file may be implemented to be forwarded externally through the second processing stream.

At this point, the storage service provided by the server end 820 may end. In 804, the server end 820 may selectively feed the P-Data-TF (RSP) back to the client end 810 to indicate the end of the storage service as a response. In 805, after the client end 810 receives the P-Data-TF (RSP), the client end 810 may send a request of A-Release-RQ for releasing the network connection to the server end 820. In 806, after receiving the A-Release-RQ, the server end 820 may feed the A-Release-RP back to the client end 810 to indicate the confirmation of releasing the network connection with the client end 810. It should further be noted that the operation 806 only represents the end of the data transmission between the client end 810 and the server end 820. The server end 820 may need to forward the DICOM file to other terminals or other servers. For example, the server end 820 may further need to establish a network connection with the real destination (e.g., the other terminals or servers), which is similar to the data transmission between the client end 810 and the server end 820, which is not repeated here.

The above descriptions may be the exemplary process when the service type is the forwarding service. Taking that there is locally stored medical image data that is the same as the unparsed medical image data as an example, a brief process of the forwarding server may further be illustrated in FIG. 10. The process of 1000 may include operations 1001-1004 as follows, which may be implemented by the processing device 400b or the server end (e.g., the server end 820).

In 1001, a DICOM file stream may be established. It should be noted that as the server end 820 includes the locally stored medical image data that is the same as the unparsed medical image data in the first processing stream, the DICOM file stream of the medical image data may be established in advance to write the medical image data to the second processing stream.

In 1003, file meta information of the DICOM file stream may be parsed. It should be noted that as the DICOM file stream is no longer a network stream, the file meta information may no longer exist in the header data. That is, in some embodiments, the DICOM file stream may not record the file meta information, and the file meta information of the DICOM file stream may be default information that needs to be inferred according to the DICOM standard (e.g., DICOM 3.0 standard).

In 1005, the parsed file meta information and the unparsed medical image data in the DICOM file stream may be written to the second processing stream. It should be noted that the second processing stream may be the socket network stream that is sent externally.

In 1007, the second processing stream may be sent externally.

According to some embodiments of the present disclosure, there is no need to write all received data sets into the memory of the image file system, which avoids excessive consumption of the memory resource. At the same time, there is no need to write all received data sets into the local cache of the image file system, and then retrieve them from the local cache, which avoids the sharp increase of I/O operations that may reduce the system processing performance. In addition, the source of the medical image data written into the second processing stream may be selected according to whether the server end (e.g., the server end 820) (which is as a forwarding transfer station) is stored with the medical image data to be forwarded, thereby improving the fault tolerance of data forwarding.

When the service type is the forwarding service, the data source of the unparsed medical image data may be the first processing stream or a local file stream. When the data source is the first processing stream, the unparsed medical image data may be obtained from the first processing stream. In such cases, the recorded data length of the medical image data in the parsed header data may be compared with the actual data length of the unparsed medical image data in the first processing stream. If the recorded data length and the actual data length are inconsistent, the unparsed medical image data may not be obtained from the first processing stream and a resend message may be fed back to the client end 810, so that the client end 810 may resend/retransmit the first processing stream. If the recorded data length and the actual data length are consistent, the unparsed medical image data may be obtained from the first processing stream, and the unparsed medical image data may be written to the second processing stream without passing the memory or the local cache. As the determination result of whether the actual data length and the recorded data length can be used to indicate whether the medical image data in the first processing stream is wrong, the determination process may avoid writing wrong medical image data to the second processing stream, thereby ensuring the data accuracy of the second processing stream in subsequent processes.

In some embodiments, the present disclosure may not limit the mode of how to process the second processing stream. For example, the second processing stream may be sent externally according to an instruction of the column storage database. The column storage database may include the C-Store. In addition, the storage service and the forwarding service may be performed concurrently (e.g., in a parallel fashion) between different client ends and server ends. According to some embodiments of the present disclosure, the concurrent amount of the C-Store instructions may be continuously increased, thereby the DICOM files may be transmitted quickly under the DICOM protocol. At the same time, the entire transmission system 100 composed of client ends and server ends may achieve higher throughput.

In some embodiments, as the C-Store database is adapted to store the DICOM files, and the data length of the C-Store database column is fixed, there is no need to consider the memory alignment in the database implementation, which avoids or reduces the situation of data crossing the boundary which may cause two memory accesses are required for one data process, so that the data element may be encoded into the most effective form without being limited by the size of the DICOM file.

It should be understood that although the operations from FIG. 7 to FIG. 10 are displayed in order according to the arrow's instructions, these operations are not necessarily performed in such order. Unless specified in the present disclosure, the implementations of the operations do not have strict order restrictions and may be performed in other orders. In some embodiments, one or more of the operations may include a plurality of sub-operations or steps, these sub-operations or steps may not necessarily be performed at the same time. For example, the plurality of sub-operations or steps may be performed at different times. As another example, the plurality of sub-operations or steps may be performed in turn with other operations or the sub-operations or steps of other operations instead of being performed in order.

FIG. 11 is a schematic diagram illustrating an exemplary process for DICOM file transmission according to some embodiments of the present disclosure. In some embodiments, the process 1100 may be implemented as a set of instructions (e.g., an application) stored in a storage device (e.g., the storage device 140, the storage device 220, and/or the storage 390). The processing device 400c (e.g., the processor 210, the CPU 340, and/or one or more modules illustrated in FIG. 4C) may execute the set of instructions, and when executing the instructions, the processing device 400c may be configured to perform the process 1100. The operations of the illustrated process presented below are intended to be illustrative. In some embodiments, the process 1100 may be accomplished with one or more additional operations not described and/or without one or more of the operations discussed. Additionally, the order of the operations of process 1100 illustrated in FIG. 11 and described below is not intended to be limiting. In some embodiments, the process 1100 may be implemented by a server end such as an image file system (e.g., the PACS and the C-Store SCU thereof). The processing device 400c may be a part of the server end. Alternatively, the process 1100 may be implemented by a client end (e.g., the terminal 130).

In 1101, the processing device 400c (e.g., the reading module 431) may obtain initial pixel label of an initial DICOM file.

In some embodiments, the initial DICOM file refers to a DICOM file of transmitted medical images. The amount of pixel data of the transmitted medical images may be relatively large, and may generally account for more than 97% of the size of the initial DICOM file. Generally speaking, the DICOM file may show a table containing a variety of tags (labels) after being parsed. Each line of the table contains an ID, a description, a type, a data length, and a label value (tag value) of a tag. For example, for a tag in a specific line, the ID of the tag may be (0010, 0010), the description of the tag may be the patient's name, the type of the tag may be PN, the data length of the tag may be 10, and the tag value of the tag may be Wu XX. Other tags, such as the patient's height, the patient's weight, and other information may also be recorded in the table of the DICOM file. For example, the DICOM file may include a tag whose ID is (7FE0,0010). The tag value of the tag (7FE0,0010) may be the pixel data of the transmitted medical image. As the pixel data accounts for more than 97% of the size of the DICOM file, the present disclosure may divide tag values of the DICOM file into pixel label values and non-pixel label values. In some embodiments, the content of the pixel label value (i.e., initial pixel data) may exist in the initial DICOM file in the form of an attachment, that is, in the form of a file including the initial pixel data. The file including the initial pixel data refers to a pixel information file of the transmitted medical image. In some embodiments, the preset transmission protocol may include a DICOM protocol (also referred to as a DICOM transmission protocol). Under the protocol, a connection pool may be established to transmit the plurality of DICOM sub-files in a parallel fashion to a target receiving end (e.g., a server end such as the C-Store SCP of the PACS, the server end 820, or a client end such as the terminal 130, etc.).

In some embodiments, the processing device 400c may read the initial pixel label value (e.g., the tag value of the tag (7FE0,0010)) in the initial DICOM file to obtain the initial pixel data, so as to divide the initial pixel data into the plurality of pixel groups, so as to facilitate the subsequent transmission of the divided initial DICOM file.

In 1103, the processing device 400c (e.g., the division module 433) may divide (or segment) the initial pixel data into a plurality of pixel groups.

In some embodiments, the processing device 400c may divide the initial pixel data equally or unequally into the plurality of pixel groups. For example, the initial pixel data may be equally divided into, e.g., 10, 15, or 20 pixel groups. As another example, the initial pixel data may be unequally divided. For instance, the first or last pixel group of the plurality of pixel groups may have different data lengths from other pixel groups of the plurality of pixel groups. In some embodiments, each of the plurality of pixel groups may be recorded with a sequence number that indicates a division (or segmentation) order of the pixel group in the initial pixel data.

In 1105 the processing device 400c (e.g., the first generation module 435) may generate a plurality of DICOM sub-files based on the plurality of pixel groups and the initial DICOM file.

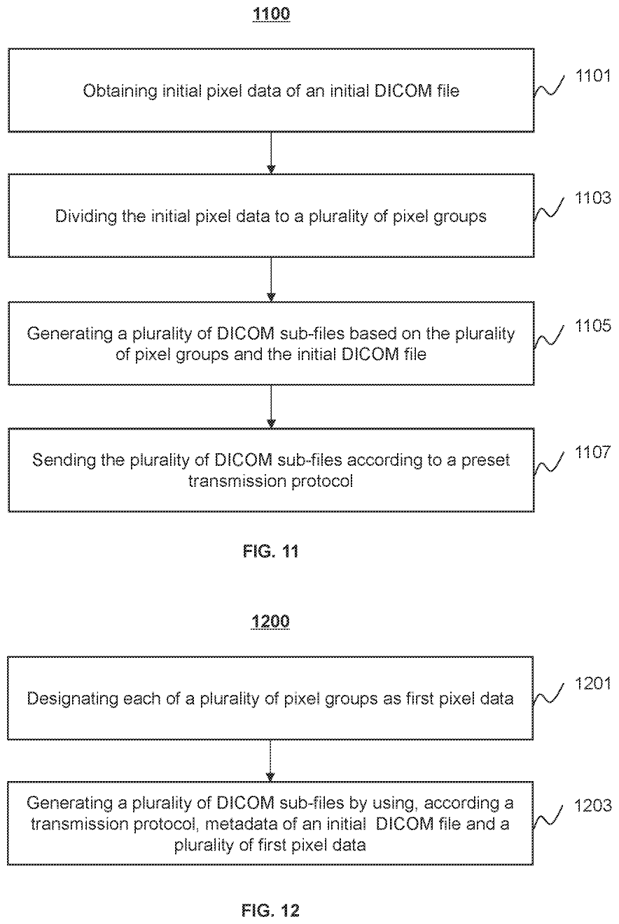

In some embodiments, the processing device 400c may generate the plurality of DICOM sub-files based on the plurality of pixel groups and the initial DICOM file according to operations of process 1200 as illustrated in FIG. 12. The process 1200 may include operations 1201 and 1203.

In 1201, the processing device 400c may designate each of the plurality of pixel groups as first pixel data.

In 1202, the processing device 400c may generate the plurality of DICOM sub-files by using, according to a transmission protocol (e.g., the DICOM protocol), metadata of the initial DICOM file and a plurality of first pixel data corresponding to the plurality of pixel groups. For example, the processing device 400c may read the non-pixel label value in the initial DICOM file to obtain the metadata of the initial DICOM file. For each of the plurality of pixel groups, the processing device 400c may generate a DICOM sub-file by combining, according to the transmission protocol, the metadata of the initial DICOM file and the pixel group.

In some embodiments, each of the plurality of the DICOM sub-files may include the divided initial pixel data (e.g., the first pixel data). As used herein, the first pixel data refers to pixel data of a DICOM sub-file. The processing device 400c may designate each of the plurality of pixel groups as one of a plurality of first pixel data of the plurality of DICOM sub-files. The processing device 400c may generate each of the plurality of DICOM sub-files by combining, according to the transmission protocol, one of the plurality of first pixel data and the metadata of the initial DICOM file, so that each DICOM sub-file may be a file with relatively complete label information (e.g., complete basis tags) to satisfy the DICOM communication protocol.

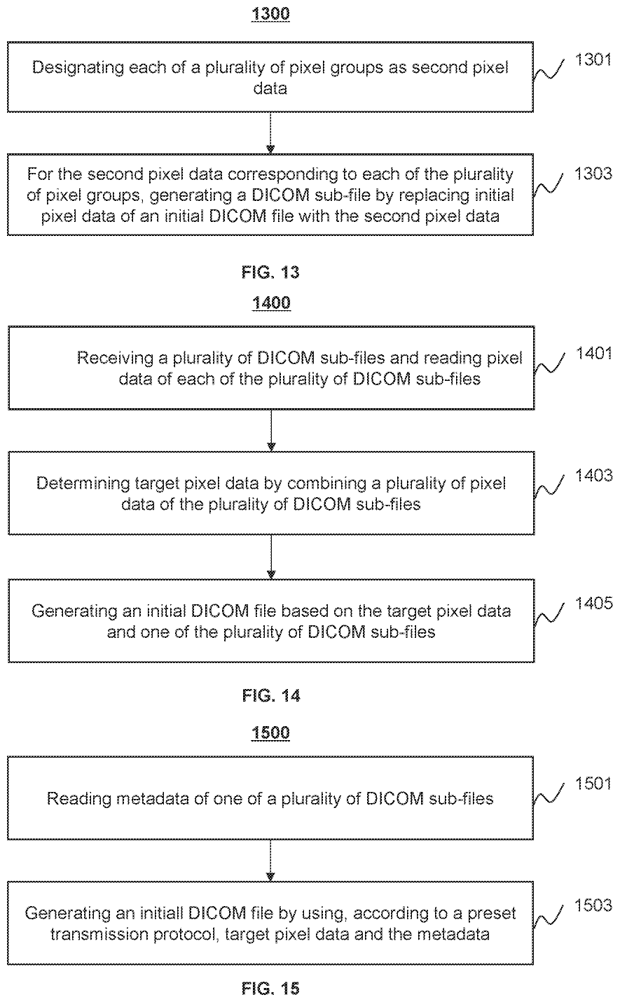

In some embodiments, the processing device 400c may generate the plurality of DICOM sub-files based on the plurality of pixel groups and the initial DICOM file according to operations of process 1300 as illustrated in FIG. 13. The process 1300 may include operations 1301 and 1303.

In 1301, the processing device 400c may designate each of the plurality of pixel groups as second pixel data.

In 1302, for the second pixel data corresponding to each of the plurality of pixel groups, the processing device 400c may generate a DICOM sub-file of the plurality of DICOM sub-files by replacing the initial pixel data of the initial DICOM file with the second pixel data.

In some embodiments, the second pixel data corresponding to each of the plurality of pixel groups may be added independently to the initial DICOM file in the form of an attachment, that is, the second pixel data corresponding to each of the plurality of pixel groups may replace the initial pixel data in the initial DICOM file, so as to obtain different DICOM sub-files. Each DICOM sub-file may contain the metadata and their corresponding second pixel data, such that each DICOM sub-file has relatively complete label information (e.g., complete basic tags) and satisfies the transmission protocol.

It should be understood that the first pixel data and the second pixel data are the same. The present disclosure uses the first and second to distinguish different embodiments. In addition, it should be noted that, in the existing technology, the DICOM file after simple physical division may not support the DICOM transmission protocol, and may not be used normally to obtain the label information (e.g., the header data); while in the present disclosure, each of the DICOM sub-files generated after division may have relatively complete label information and supports the DICOM transmission protocol. The receiving end may normally obtain label information of each DICOM sub-file, which helps to complete the DICOM file transmission, storage, and display of the medical images.

In 1107, the processing device 400c (e.g., the sending module 437) may send/transmit the plurality of DICOM sub-files according to a preset transmission protocol (e.g., the DICOM protocol).

In some embodiments, each of the plurality of DICOM sub-file may be sent/or transmitted via a data stream (e.g., a network stream such as the first data stream as described in FIG. 5 or the first processing stream as described in FIG. 7) via a network (e.g., the network 120) to a server end (e.g., the processing device 400a, 400b, or 400d, the C-Store SCP, or the server end 820). That is, the plurality of DICOM sub-files may be transmitted in a parallel fashion. During the transmission of the plurality of DICOM files, one or more of the plurality of data streams corresponding to the plurality of DICOM files may be interrupted (e.g., which may be caused by a network factor, a bandwidth factor, a human factor, etc.). The processing device 400c may record the one or more of the plurality of data streams and re-transmit the one or more of the plurality of data streams. That is, data streams that have not been recorded may be not re-transmitted. In some embodiments, as described in FIGS. 5 and 7, a DICOM sub-file may be transmitted by multiple segments of a data stream. When the data stream is interrupted during transmission, the processing device 400c may record an interruption position (e.g., a specific segment of the data stream) and re-transmit segments of the data stream after the interruption position. That is, segments of the data stream before the interrupt position may not re-transmit.

FIG. 14 is a schematic diagram illustrating an exemplary process for DICOM file storage according to some embodiments of the present disclosure. In some embodiments, the process 1400 may be implemented as a set of instructions (e.g., an application) stored in a storage device (e.g., the storage device 140, the storage device 220, and/or the storage 390). The processing device 400d (e.g., the processor 210, the CPU 340, and/or one or more modules illustrated in FIG. 4D) may execute the set of instructions, and when executing the instructions, the processing device 400c may be configured to perform the process 1400. The operations of the illustrated process presented below are intended to be illustrative. In some embodiments, the process 1400 may be accomplished with one or more additional operations not described and/or without one or more of the operations discussed. Additionally, the order of the operations of process 1400 illustrated in FIG. 14 and described below is not intended to be limiting. In some embodiments, the process 1400 may be implemented by a server end such as the image file system (e.g., a local file system such as the PACS or the C-Store SCP thereof). The processing device 400d may be a part of the server end. Alternatively, operations of the process 1400 may be implemented by the processing device 120a or 120b.

In 1401, the processing device 400d (e.g., the receiving module 441) may receive a plurality of DICOM sub-files (e.g., from a client end such as the terminal 130 or the client end 810) and read pixel data (e.g., first pixel data/second pixel data) of each of the plurality of DICOM sub-files. The pixel data of each of the plurality of DICOM sub-files may carry a corresponding sequence number that indicates an initial division order of the pixel data.

In 1402, the processing device 400d (e.g., the combination module 443) may determine target pixel data by combining/merging a plurality of pixel data of the plurality of DICOM sub-files. For example, the plurality of pixel data may be combined or merged according to their corresponding sequence numbers.

In 1403, the processing device 400d (e.g., the second generation module 445) may generate an initial DICOM file based on the target pixel data and one of the plurality of DICOM sub-files. The initial DICOM file may be used to generate the plurality of DICOM sub-files.

In some embodiments, when the plurality of DICOM sub-files (e.g., 10 DICOM sub-files) are received, pixel data of each DICOM sub-file may be read to obtain 10 pixel data. The 10 pixel data may be combined according to sequence numbers corresponding to the 10 pixel data to obtain the target pixel data. The initial DICOM file corresponding to the 10 DICOM sub-files may be generated based on the target pixel data and one of the 10 DICOM sub-files. In some embodiments, a count (or number) of the plurality of DICOM sub-files received may be determined according to the actual count (or number) of divisions.

In some embodiments, the processing device 400d may generate the initial DICOM file based on the target pixel data and one of the plurality of DICOM sub-files according to operations of process 1500 as illustrated in FIG. 5. The process 1500 may include operations 1501 and 1502.

In 1501, the processing device 400b may read metadata (e.g., all the non-pixel label values) in one of the plurality of DICOM sub-file.

In 1502, the processing device 400b may generate the initial DICOM file by using, according to the preset transmission protocol, target pixel data and the metadata.

It should be noted that the target pixel data refers to the complete initial pixel data of the initial DICOM file that is determined after the combination. After receiving all the DICOM sub-files, the processing device 400b may read/obtain the metadata of any one of the DICOM sub-files. Then, the target pixel data and the metadata in any one of the DICOM sub-files may be combined according to the preset transmission protocol (e.g., the DICOM protocol) to generate the complete initial DICOM file, thereby the transmitted medical images can be completely displayed.

In some embodiments, the processing device 400d may generate the initial DICOM file by designating the target pixel data as the pixel data of any one of the DICOM sub-files. For example, the complete initial DICOM file may be generated by replacing the pixel data of any one of the DICOM files with the target pixel data).

Different from the existing technology, according to some embodiments of the present disclosure, the initial pixel data (e.g., the tag value of the tag ID (7FE0,0010)) in the initial DICOM file may be read and obtained and the initial pixel data may be divided into the plurality of pixel groups, so as to facilitate the subsequent transmission of the divided initial DICOM file. According to the plurality of pixel groups and the initial DICOM file, the plurality of DICOM sub-files may be generated, which ensures that each DICOM sub-file can have complete basic tags and information thereof to satisfy the transmission protocol (e.g., the DICOM protocol). By sending the plurality of DICOM sub-files through the preset transmission protocol, the image file system may achieve successful transmission of the initial DICOM file by the transmission of the DICOM sub-files, which helps the target receiving end to display the transmitted medical images normally. It should be noted that compared with the initial DICOM file, despite the different integrity of the pixel data, the header data/metadata of each DICOM sub-file may be completely consistent with that of the initial DICOM file, so as to support the DICOM protocol for transmission and storage.

In addition, the target receiving end may receive each of the DICOM sub-files and read (e.g., obtain) the pixel data of each of the DICOM sub-files. Then pixel data of each of the DICOM sub-files may be combined/merged to obtain the target pixel data. Then, the complete initial DICOM file may be generated quickly based on any one of the DICOM sub-files and the target pixel data, so as to smoothly store the initial DICOM file and/or normally display the transmitted medical image.

It should be noted that the above descriptions are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added to or omitted from a specific process. In some embodiments, an operation of a specific process may be divided into two or more sub-operations. In some embodiments, operations 1403 and 1405 may be omitted in some situations (e.g., the received DICOM sub-file needed to be forwarded to other client ends or other server ends). For example, the processing device 400d may directly store the plurality of DICOM sub-files without performing combination and generation operations.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer-readable media having computer-readable program code embodied thereon.

A non-transitory computer-readable signal medium may include a propagated data signal with computer readable program code embodied therein, for example, in baseband or as part of a carrier wave. Such a propagated signal may take any of a variety of forms, including electromagnetic, optical, or the like, or any suitable combination thereof. A computer-readable signal medium may be any computer-readable medium that is not a computer-readable storage medium and that may communicate, propagate, or transport a program for use by or in connection with an instruction execution system, apparatus, or device. Program code embodied on a computer-readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object-oriented programming language such as Java, Scala, Smalltalk, Eiffel, JADE, Emerald, C++, C#, VB. NET, Python or the like, conventional procedural programming languages, such as the “C” programming language, Visual Basic, Fortran, Perl, COBOL, PHP, ABAP, dynamic programming languages such as Python, Ruby, and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof to streamline the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A method for digital imaging and communications in medicine (DICOM) file processing, comprising:
receiving a request for processing a DICOM file, the DICOM file including data of metadata and pixel data;
parsing at least part of the metadata of the DICOM file; and
writing the data of the DICOM file to one or more data streams based on the parsed metadata.

2. The method of claim 1, wherein the writing data of the DICOM file to one or more data streams based on the parsed metadata includes:
obtaining a size of the pixel data of the DICOM file from the parsed metadata; and
writing the data of the DICOM file to the one or more data streams based on whether the size of the pixel data exceeds a threshold.

3. The method of claim 2, wherein the threshold is determined at least based on the size of cache of a system executing the method.

4. The method of claim 2, wherein the writing the data of the DICOM file to the one or more data streams based on whether the size of the pixel data exceeds a threshold includes:
in response to determining that the size of the pixel data exceeds the threshold, writing the parsed metadata of the DICOM file and the pixel data of the DICOM file to one of the one or more data streams, wherein at least part of the pixel data of the DICOM file is not parsed.

5. The method of claim 2, wherein the writing the data of the DICOM file to the one or more data streams based on whether the size of the pixel data exceeds a threshold includes:
in response to determining that the size of the pixel data does not exceed the threshold,
parsing the pixel data of the DICOM file; and
writing the parsed metadata of the DICOM file and the parsed pixel data of the DICOM file to one of the one or more data streams.

6. The method of claim 1, further comprising:
segmenting the pixel data of the DICOM file into a plurality of pixel groups; and
generating a plurality of DICOM sub-files based on the plurality of pixel groups respectively and the DICOM file.

7. The method of claim 6, further comprising:
writing data of the plurality of DICOM sub-files to the one or more data streams.

8. The method of claim 7, further comprising:
transferring the plurality of DICOM sub-files or the one or more data streams in a parallel fashion.

9. The method of claim 6, wherein the segmenting the pixel data into a plurality of pixel groups includes:
obtaining an optimal size for segmentation; and
segmenting the pixel data into the plurality of pixel groups based on the optimal size.

10. The method of claim 9, wherein the optimal size is adjustable in real time.

11. The method of claim 6, wherein each of the plurality of pixel groups carries a sequence number that indicates a segmentation order of the pixel group in the pixel data.

12. A method for digital imaging and communications in medicine (DICOM) file processing, comprising:
receiving a request for processing a DICOM file;
obtaining a first data stream including data of the DICOM file;
parsing metadata of the DICOM file from the first data stream; and
writing the parsed metadata of the DICOM file and pixel data of the DICOM file to a second data stream, wherein the pixel data is not parsed.

13. The method of claim 12, wherein the second data stream is a file stream, and the processing a DICOM file includes storing the DICOM file in a local file system.

14. The method of claim 12, wherein the second data stream is a network stream, and the processing a DICOM file includes transmitting the DICOM file to an external system.

15. The method of claim 11, wherein the metadata of the DICOM file includes a recorded data length of the pixel data of the DICOM file, and the method further comprises:
determining an actual data length of the pixel data of the DICOM file; and
obtaining the pixel data from the first data stream based on whether the recorded data length is equal to the actual data length.

16. A method for digital imaging and communications in medicine (DICOM)file processing, comprising:
obtaining a DICOM file;
obtaining pixel data of the DICOM file;
segmenting the pixel data into a plurality of pixel groups;
generating, based on the plurality of pixel groups and the DICOM file, a plurality of DICOM sub-files.

17. The method of claim 16, further comprising:
transferring the plurality of DICOM sub-files in a parallel fashion.

18. The method of claim 16, wherein the generating, based on the plurality of pixel groups and the DICOM file, a plurality of DICOM sub-files includes:
parsing metadata of the DICOM file; and
for each of the plurality of pixel groups,
generating a DICOM sub-file corresponding to the pixel group by using, according to a DICOM protocol, the parsed metadata of the DICOM file and the pixel group.

19. The method of claim 16, wherein the generating, based on the plurality of groups and the DICOM file, a plurality of DICOM sub-files includes:
for each of the plurality of pixel groups, determining a DICOM sub-file corresponding to the pixel sub-data by using the pixel group as pixel data of the DICOM sub-file with the pixel sub-data.

20. The method of claim 16, wherein the segmenting the parsed pixel data into a plurality of pixel groups includes:
obtaining an optimal size for segmentation; and
segmenting the parsed pixel data into the plurality of pixel groups based on the optimal size.
